(19) United States
(12) Patent Application Publication
(10) Pub. No.: US 2022/0051586 A1
(43) Pub. Date: Feb. 17, 2022

(54) SYSTEM AND METHOD OF GENERATING CONTROL COMMANDS BASED ON OPERATOR'S BIOELECTRICAL DATA

(71) Applicant: I-BRAINTECH LTD, Ramat Gan (IL)

(72) Inventors: Lev STANKEVICH, Saint-Petersburg (RU); Natalia SHEMYAKINA, Saint-Petersburg (RU); Zhanna NAGORNOVA, Saint-Petersburg (RU); Filipp GUNDELAKH, Saint-Petersburg (RU); Aleksandra CHEVYKALOVA, Saint-Petersburg (RU)

(21) Appl. No.: 17/279,313

(22) PCT Filed: Sep. 24, 2019
(86) PCT No.: PCT/IB2019/058100

§ 371 Date: Mar. 24, 2021

(30) Foreign Application Priority Data
Sep. 24, 2018 (RU) 2018133658

(51) Int. Cl. G09B 19/00; G06F 3/01; G06K 9/00

(52) U.S. Cl. CPC G09B 19/003; A63F 13/212; G06K 9/0053; G06F 3/015

(57) ABSTRACT

The technical solution relates to control systems, more particularly to systems and methods of generating control commands based on operator's bioelectrical data. One more technical result of the present technical solution is the increase of identification accuracy of the Operator's actions. One more technical result of the present technical solution is the improvement of identification of the Operator's actions due to the elimination of artefacts from the Operator's bioelectrical data.

### TECHNICAL FIELD OF THE INVENTION

The technical solution relates to control systems, more particularly to systems and methods of generating control commands based on operator's bioelectrical data.

### BACKGROUND OF THE INVENTION

Currently, computing technologies are much involved in everyday life. The number of various computers, home appliance controllers has exceeded the Earth's population many times and continues to increase at an accelerating pace. Many facets of people's lives are automatized and computerized, including road traffic control, online shopping, control and automated configuration of smart home devices according to users' demands etc.

Together with the development of computing technologies, various methods to control the above-mentioned devices have also been updated, including data input from the keyboard, the use of styluses and touch pads, recognition of visual images and speech. In recent years, control methods with intelligent commands, decoding of bioelectrical activity of the brain (analysis, classification and detection of specific information elements; for this, the human's brain activity of various types is detected, including electroencephalographic signals, hemodynamic response etc.)

One of the lines of development of computing technologies is the use of computing technologies for after-care of people, who have completely or partially lost the opportunity to live a productive life (e.g. who suffered a blood stroke, a limb loss, a traumatic brain injury etc.) Various methods of human-computer interaction (including a direct control with intelligent commands) are used for after-care of such people.

The main complications in the implementation of systems with the use of the above-mentioned technologies, include:
a challenge to collect data on a person with the accuracy, required to operate the system; this challenge relates to the features of sensors, used for the above-mentioned data collection (for example, the sensitivity and correct positioning of sensors);
a selection of individual data, informative for every person (e.g., EEG signals, corresponding to imagined moves of two people, can be very different);
variability of collected data according to time;
a challenge to process data (for example, data processing, requiring either considerable time expenses, or the use of considerable computing resources).

For example, Publication US2017347906 describes the technology of brain activity analysis and performance of some actions based on the analysis. To collect data on brain activity, a system of sensors is used, which are fixed on the user's head. The sensors detect the modifications of electromagnetic potential, which is created with the brain's bioelectrical activity, and transform the acquired data into digital data. This digitized data is analyzed and assigned some pre-configured patterns (images) of brain activity and, depending on the similarity of the analyzed data on brain activity and on specific images, the decision on the type of moves, made by the user. The advantage of the technology, described in the publication, is the possibility to detect user's actions based on their brain activity; the disadvantage is the impossibility to adapt the technology to a particular user, due to which the accuracy of detection of the user's action can be low. Moreover, the above-mentioned technology lacks the implementation of feedback, when in addition to image recognition based on user's brain activity, the user is provided with feedback depending on performed actions (on images), which can cause the modifications in the brain's bioelectrical activity and can have corrective and optimizing effect.

The technology, described above, is adequately used with the tasks on recognition of actions, made or imagined by the user; however, the technology, described above, adequately recognizes only a small and limited number of the user's actions, having low productivity, which makes it difficult to give corrective feedback in real time.

The given technical solution allows solving the task to generate control commands with external hardware and software based on the operator's bioelectrical data.

### SUMMARY OF THE INVENTION

The technical solution is designed for generating control commands with external means (devices) based on the Operator's bioelectrical data.

One more technical result of the present technical solution is the increase of identification accuracy of the Operator's actions.

One more technical result of the present technical solution is the improvement of identification of the Operator's actions due to the elimination of artefacts from the Operator's bioelectrical data.

One more technical result of the present technical solution is the improvement of identification of the Operator's actions due to overtraining the model, used to identify the Operator's actions.

One more technical result of the present technical solution is the performance of after-care activity by using neurofeedback.

These results are achieved by using a method of real time rehabilitation and training comprising steps of: (a) forming a virtual domain further comprising an operator's character; (b) forming a task to be performed by an operator; (c) collecting operator's bioelectrical data; (d) detecting characteristic features of the collected bioelectrical data by means of artificial intelligence; (e) defining an action pattern according to the detected characteristic features; (f) generating a control command for the virtual domain based on the defined action pattern which is displayed to the operator; (g) evaluating execution performance of the operator's action; (h) evaluating operator's task execution performance; (i) providing a feedback to the operator's executed task in real time; and (j) performing a calibration for the operator.

In another particular embodiment of the method, the step of collecting the operator's bioelectrical data further comprise collecting at least one of the following: (a) an operator's electroencephalogram being a set of electroencephalographic signals of an operator's nervous system; the set is characterized by a signal registration time of the electroencephalographic signals and a signal amplitude of the electroencephalographic signals; and (b) an operator's electromyogram being a set of electromyographic signals of an operator's muscular system; the set is characterized with a signal registration time of the electromyographic signals and a signal amplitude of the electromyographic signals.

In another particular embodiment of the method, the step of extracting at least one characteristic features collected from bioelectrical data is performed by means of at least one of the following: (a) a trained model for feature extraction, and (b) a set of feature extraction rules.

In another particular embodiment of the method, the step of the at least one characteristic feature is selected from the group consisting of: a spectral characteristic, a time characteristic, a wavelet decomposition characteristic, a spatiotemporal characteristic and any combination thereof.

In another particular embodiment of the method, the step of evaluating execution performance comprises evaluating conformity of the state of the virtual object after performing the operation by the operator at the virtual object; the conformity is evaluated in comparison with a predesigned resultant state of the virtual object after performing the operator's action.

In another particular embodiment of the method, the step of evaluating operator's task execution performance further comprises evaluating a number of errors of performing the action by the operator at the virtual object; the errors are indicated when the action is performed by the operator at the virtual object with an execution performance lower than a preconfigured value.

In another particular embodiment of a computer-implemented system for generating control commands based on the operators' bioelectrical data comprises: (a) a processor; (b) a memory storing instructions which, when executed by the processor, direct the processor to: (i) collecting operator's bioelectrical data and transferring the collected data: (ii) extracting at least one characteristic feature from collected bioelectrical data by means of at least one of the following: (1) a trained model for feature extraction based on machine learning, and (2) a set of feature extraction rules; the at least one characteristic feature is selected from the group consisting of: a spectral characteristic, a time characteristic, a wavelet decomposition characteristic, a spatiotemporal characteristic and any combination thereof; (c) defining an action pattern according to the extracted characteristic features by means of artificial intelligence; the action pattern being a numerical value, which characterizes the possibility of belonging the operator's collected bioelectrical data to the action; (d) generating a control command based on an action pattern.

In another particular embodiment of the system, the operator's bioelectrical data further comprise at least one of the following: (a) an operator's electroencephalogram being a set of activity signals of an operator's nervous system; the set is characterized by a signal registration time and a signal amplitude thereof; and (b) an operator's electromyogram being a set of activity signals of an operator's muscular system; the set is characterized by a signal registration time and a signal amplitude thereof.

In another particular embodiment of the computer-implemented system, the instructions comprise extracting at least two samples from the collected bioelectrical data; each the sample is a set of data describing a single image of an operator's move.

In another particular embodiment of the computer-implemented system, the action pattern is defined by a two-level committee of local classifiers comprising a lower level and an upper level; the lower level further comprises a combination of at least one classifier based on a support vector machine and at least one artificial neural network; the upper level further comprises at least one artificial neural network.

In another particular embodiment of the computer-implemented system, the artificial neural network of the upper level of committee of local classifiers is trained on a dataset comprising solutions for each of the local classifier of the lower level.

In another particular embodiment of the computer-implemented system, the memory comprises an instruction of analyzing and transforming the collected data. The aforesaid instruction of analyzing and transforming the collected data further comprises: (a) applying high and low frequency filters; (a) removing a network noise by applying at least one of band elimination and band-pass filters, (b) filtering filtered EEG signals; (c) transforming the EEG signal into mean, weighed mean composition, current source density, topographies of independent components.

In another particular embodiment of the computer-implemented system, the instructions comprise an instruction of forming of an image of the action and displaying thereof to the operator.

In another particular embodiment of the computer-implemented system, the instructions comprise simultaneously accounting for the properties of a two-level committee of local classifiers; the two-level committee comprises a lower level further comprising at least two artificial neural networks and at least of two support vector machines, and an upper level comprising an artificial neural network combining classification results of the lower level.

In another particular embodiment of a computer-implemented method of generating control commands based on operator's bioelectrical data comprises steps of: (a) providing a computer-implemented system for generating control commands; the system comprising a processor and a memory for storing instructions for implementing the method; (b) collecting operator's bioelectrical data; (c) extracting at least one characteristic features from collected bioelectrical data by means of at least one of the following: (i) a trained model for feature extraction, (ii) a set of feature extraction rules; the at least one characteristic feature selected from the group consisting of: a spectral characteristic, a time characteristic, a wavelet decomposition characteristic, a spatiotemporal characteristic and any combination thereof; (d) defining an action pattern according to the extracted features by means of artificial intelligence; the action pattern being a numerical value, which characterizes the possibility of belonging the collected bioelectrical data to the configured imagine action of the operator; and (e) generating a control command based on an action pattern.

In another particular embodiment, the computer-implemented method comprises extracting at least two samples from the collected bioelectrical data; each the sample is a set of data corresponding to a single image of an operator's move.

In another particular embodiment, the computer-implemented method comprises steps of analyzing and transforming the collected data; the steps of analyzing and transforming the collected data comprises at least one of the following: (a) applying high and low frequency filters; (b) removing a network noise is removed, using at least band elimination and band-pass filters, (c) filtering EEG signals; (d) transforming EEG signals into mean, weighed mean composition, current source density, topographies of independent components.

In another particular embodiment, the computer-implemented method comprises an instruction of forming an image of the action and displaying thereof to the operator.

In another particular embodiment, a computer-implemented system for evaluating execution performance of an operator based on the operator's bioelectrical data comprises: (a) a processor; (b) a memory storing instructions which, when executed by the processor, direct the processor to: (i) generating a virtual domain comprising at least one virtual object characterized by a feature selected from the group consisting of: a position in the virtual domain, a dimension, a color, an interaction rule for the virtual domain, a rule of changing a state of the virtual object depending on an operator's action in the virtual domain; (ii) at least one action to be performed by the operator and related to at least one virtual object; (b) an actuator configured for performing the at least one operator's action under the generated control command in the virtual domain; the memory further comprises instructions to: (1) Evaluating conformity of the state of the virtual object after performing an action at the virtual object by the operator to a predesigned resultant state of the virtual object after performing the operator's action; (2) evaluating a number of errors of performing the action by the operator at the virtual object.

In another particular embodiment, the errors are indicated when the action is performed by the operator at the virtual object with an execution performance lower than a preconfigured value.

In another particular embodiment, the computer-implemented system comprises the virtual domain, the virtual objects in the virtual domain and the actions performed by the operator in the virtual domain are visualized.

In another particular embodiment, the operator's operation comprises a change of the state of the at least one virtual object with the at least one operator's action.

In another particular embodiment, the change of the state of the at least one virtual object is performed by the operator at least one of the following conditions: (a) within a preconfigured time period, (b) with the preconfigured number of tries.

In another particular embodiment, a computer-implemented method of evaluating execution performance of an operator based on the operator's bioelectrical data comprising steps of: (a) providing a computer-implemented system for evaluating execution performance of an operator based on the operator's bioelectrical data; the system comprising a processor and a memory for storing instructions for implementing the method; (b) generating a virtual domain further comprising at least one virtual object; a state of the virtual object having a characteristic selected from the group consisting of: a position in the virtual domain, a dimension, a color, an interaction rule for the virtual domain, a rule of changing the object depending on the operator's action in the virtual domain and any combination thereof; at least one action related to at least one virtual object to be performed by the operator; (c) collecting operator's bioelectrical data; (d) generating at least one control command based on the collected the operator's bioelectrical data; (e) performing at least one operator's action under a generated control command in the virtual domain; (f) evaluating conformity of the state of the virtual object after performing the operation by the operator at the virtual object to a predesigned state of the virtual object after performing the operator's action; (g) evaluating a number of errors of performing the action by the operator at the virtual object.

In another particular embodiment of the computer-implemented method, the errors are indicated when the action is performed by the operator at the virtual object with an execution performance lower than a preconfigured value.

In another particular embodiment, a computer-implemented method, the virtual domain, the virtual objects in the virtual domain and the actions performed by the operator at the virtual domain are visualized.

In another particular embodiment, a computer-implemented method, the operator's operation comprises a change of the state of at least one of the virtual object with at least one of the operator's action.

In another particular embodiment, a computer-implemented method, the change of the state of the virtual object is performed by the operator at least one of the following conditions: (a) within a preconfigured time period, and (b) with a preconfigured number of tries.

### BRIEF DESCRIPTION OF DRAWINGS

FIG. 1 depicts a flowchart of the system of generating control commands based on Operator's bioelectrical data, in accordance with at least one non-limiting embodiment.

FIG. 2 depicts a flowchart of the method of generating control commands based on Operator's bioelectrical data, in accordance with at least one non-limiting embodiment.

FIG. 3 depicts a flowchart of the system of generating control commands based on Operator's bioelectrical data, in accordance with at least one non-limiting embodiment.

FIG. 4 depicts a flowchart of the method of generating control commands based on Operator's bioelectrical data, in accordance with at least one non-limiting embodiment.

FIG. 5 depicts a flowchart of the task execution performance evaluation system based on Operator's bioelectrical data, in accordance with at least one non-limiting embodiment.

FIG. 6 depicts a flowchart of the task execution performance evaluation method based on Operator's bioelectrical data, in accordance with at least one non-limiting embodiment.

FIG. 7 depicts a general workflow of the visual game framework with the use of the system of generating control commands based on Operator's bioelectrical data, in accordance with at least one non-limiting embodiment.

FIG. 8 depicts a block diagram of an algorithm for the main section of the visual game framework with the use of the system of generating control commands based on Operator's bioelectrical data, in accordance with at least one non-limiting embodiment.

FIG. 9 depicts an example of a sample processing cycle, in accordance with at least one non-limiting embodiment.

FIG. 10 depicts an example of Operator's interaction with the visual game framework using the system of generating control commands based on Operator's bioelectrical data, in accordance with at least one non-limiting embodiment.

FIG. 11 is an example of the amplitude frequency response of the band elimination filter.

FIG. 12 is an example of EEG-signals.

FIG. 13 is an example of EEG with artefacts.

FIG. 14 depicts an example of characteristic feature classification system, in accordance with at least one non-limiting embodiment.

FIG. 15 depicts an example of the flowchart of Operator's after-care system, in accordance with at least one non-limiting embodiment.

FIG. 16 depicts an example of the flowchart of Operator's after-care method, in accordance with at least one non-limiting embodiment.

FIG. 17 depicts an example of the flowchart of the classifiers' committee, in accordance with at least one non-limiting embodiment.

FIG. 18 depicts an example of general-purpose computing system, in accordance with at least one non-limiting embodiment.

Though the technical solution can have various modifications and alternative forms, the characteristic features, shown as examples in the drawings, will be given a detailed description. It will be appreciated that the purpose of the description is not the limitation of the technical solution by its specific claim. Vice versa, the purpose of the description is the breadth of all changes and modification within the given technical solution according to the patent claim.

### DETAILED DESCRIPTION OF THE INVENTION

The object and features of the given technical solution, and the methods to achieve these objects and features, will become evident by referencing approximate embodiments. However, the present technical solution is not limited by approximate embodiments, disclosed below; it can be embodied in various forms. The essence described is none but the details, required by specialists in the field of technology in the comprehensive representation of the technical solution, and the given technical solution is defined by the scope of the attached patent claim.

Let us introduce a multiplicity of definitions and notions, which will be used to describe the embodiments of the technical solution.

The term “bioelectrical data” hereinafter refers to bioelectrical signals of the activity of the human's brain and nervous system.

The term “wavelet decomposition” hereinafter refers to an integral decomposition, which is an outline of Wavelet function with the signal. A wavelet decomposition transforms the signal from its time representation into time-and-frequency representation. A wavelet decomposition of signals is the summary of spectral analysis.

The term “wavelets” hereinafter refers to a general name of mathematical functions of a definite form, which are local in time and frequency and in which all the functions come out of one basic function by changing (translating, stretching) it.

The term “discrete Fourier transformation” hereinafter refers to one of Fourier transformations, widely used in digital signal processing algorithms, as well as in other spheres, related to frequency analysis in a discrete (e.g., digitized analog) signal. Discrete Fourier transformation requires a discrete function as an input. Such functions are often made by discretization (sampling values from continuous functions).

Direct transformation is given by:

$${X}_{k} = {\sum }_{n = 0}^{N - 1} {x}_{n} {e}^{- \frac{2 π i}{N} kn}$$

Inverse transformation is given by:

$${x}_{n} = {\sum }_{k = 0}^{N - 1} {X}_{k} {e}^{\frac{2 π i}{N} kn}$$

The term “Brain-computer interface (BCI)” hereinafter refers to a system of generating control commands based on Operator's bioelectrical data.

The term “Artificial neural network hereinafter refers to a set of neurons, united into a network by connecting neuron inputs of one layer with neuron outputs of another layer; at that, the neuron inputs of the first layer are the inputs of the whole neural network, and the neuron outputs of the last layer are the outputs of the whole neural network.

From the point of view of machine learning, the use of neural networks is a special case of pattern recognition methods, discriminatory analysis, classification methods etc.

The term “Machine learning (ML)” hereinafter refers to a class of artificial intelligence methods, the particularity of which is not a direct solution of the task but learning in the process of implementing solutions of numerous similar tasks. For this, the methods mathematical statistics, numeric procedures, optimization, theory of probability, theory of graphs, various methods of digital data operations.

There are three types of learning:
learning from examples or inductive learning, based on the detection of empirical regularities in data;
deductive learning, considering expert knowledge formalization and transfer of this knowledge as a knowledge data base;
reinforcement learning based on a rule-of-thumb methods with motivation to correct actions in the current situation.

The term “Support vector machine method (SVM)” hereinafter refers to a set of similar learning algorithms with a teacher, used for classification tasks and regression analysis. It belongs to the family of linear classifications. A special feature of the Support Vector Machine is a continuous decrease of empiric classification error and increase of margins, that is why the method is also known as a maximum-margin classification method.

The main idea of the method is the transfer of original vectors into the space of a higher dimension and the search of the separating hyperplane with the maximal margin in this plane. Two parallel hyperplanes are formed at both sides of the hyperplane dividing the classes. The separating hyperplane will be the hyperplane, which maximized the distance to the two parallel hyperplanes. The algorithm operates on the suggestion that the greater difference or distance there is between these parallel hyperplanes, the less will be the average classification error.

The term “Fourier transformation” hereinafter refers to an operation, comparing one function of real variable to another function of real variable. This new function describes coefficients (“amplitudes”) at fracturing of the original function into simples, which are harmonic vibrations of various frequency (like a chord, which can be expressed as the sum of its musical sounds). Fourier transformation of / function real variable is integral and is set by the following formula:

$$\hat{f} ( w ) = \frac{1}{\sqrt{2 π}} {{\int }^{∞}}_{- ∞} f ( x ) {e}^{- ixw} dx .$$

At the creation of a system of generating control commands based on the Operator's bioelectrical data under EEG signal of imaginary moves, the key development tasks are the detection of control signal, the detection of its features and the classification of these features in real time. The solution of these tasks is the necessary step to create the applicable after-care system based on the system of generating control commands based on the Operator's bioelectrical data.

The special feature of EEG, registered from the head (scalp) surface is its “lower spatial resolution (about a square centimeter) as compared with electrocorticogram data (registration of bioelectrical activity from the brain surface) and magnetoencephalogram, the spatial resolution of which can be a few square millimeters”. “When transmitting the brain tunic, skull and scalp, the amplitude of bioelectrical signals considerably decreases (especially for the high-frequency component); the presence of tunic with various specific resistance leads to the blurring of the potential through the scalp; thus, the head surface emits not only the signal from the field closer to the electrode, but also from the farther field, when the signal generator is distanced from the registering electrode by extensional current conduction by the brain and signal transmitting in brain tunic”. On the one hand, it prevents a clear signal localization; on the other hand, it can be partially overcome with signal spatial filtration and source detection with main or independent component method.

Based on the conducted empiric studies, the best classification results of single samples of EEF signals, particularly, features, calculated as specific signal characteristics in time domain (e.g., of such its features as length and area under the curve), which can be reached by using transformations to the current source density and/or independent component methods. At this, the best results are achieved for the classification of the curve length.

Beside the specific of the registered signal, to develop the applicatory real time BCI platform it is necessary to overcome the existing technology barriers to couple EEG registration hardware systems and automated data processing software. In the given project, such barriers are overcome with the use of a wireless electroencephalograph with data transfer to LSL (Lab Streaming Layer, an international standard for batch data transmission, including bioelectrical activity).

The given approach provides the possibility to acquire data with minimal time delay and does not require any special software by external developers. However, in this case there is a necessity to implement authoring software complex of the full cycle of registration, synchronization, data processing and data analysis. The main element of the developed EEG signal registration system is the unit for eliminating hardware delays and for synchronizing timer clocks.

One more requirement to the practical implementation of the BCI as part of the after-care complex is providing the user with feedback from the system, which forms neurofeedback. At this, each intelligent command must have a matching observed response from the system, and time delay to calculate this response must be low (max. 500 ms) so that these events would be closely related in the user's mind.

For this, it is necessary that EEG signal registration and recognition systems would successfully interact and provide the output of the results with the delay, which does not exceed time noticeable by the user. In whole, the time required to give BCI response, in any case must be lower than the period of performance of a single imagined move.

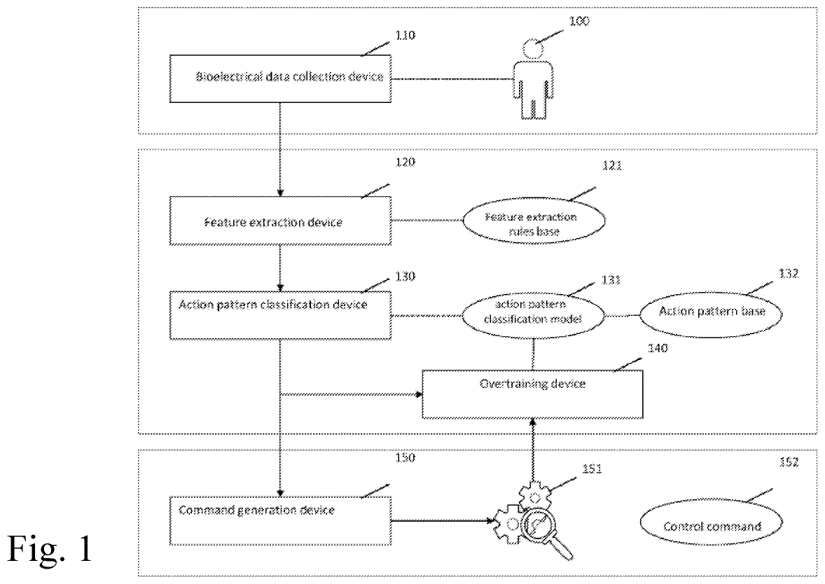

FIG. 1 is a flowchart of the system of generating control commands based on Operator's bioelectrical data.

A flowchart of the system of generating control commands based on Operator's bioelectrical data consists of Operator 100, bioelectrical data collection means (device) 110, feature extraction means (device) 120, feature extraction rules base 121, action pattern classification means (device) 130, action pattern classification model 131, action pattern base 132, control command generation means (device) 150, external control means (device)s 151, control command base 152.

Operator 100 is a person, remotely controlling external control means (device)s 151 with the described system.

For example, the following can act as operator 100:
a patient, using the described system for after-care (for example, after the previous stroke or limb loss);
a person, using the described system as a game controller to control the game;
a person, using the described system as a system to train cognitive abilities (attention, memory, learning ability etc.).

The bioelectrical data collection means (device) 110 is designed to:
collect bioelectrical data of operator 100;
transfer the collected data to feature extraction means (device) 120.

In one of the embodiments, the collection of bioelectrical data is performed at least:
non-invasively, with sensors, located remotely from operator 100 or fixed on operator 100;
invasively, with sensors, implanted into operator 100;
with a combined method, i.e. using both invasive and non-invasive method of bioelectrical data collection.

For example, to collect data on brain's activity (electroencephalogram, EEG) of operator 100, a set of sensors (electrodes), attached to the head of operator 100, or located at a small distance from the head of operator 100, can be used (for example, a set of sensors, fixed into a head-piece).

In another example, in case a constant operation is required, for the comfort of operator 100 (for instance, in case of disability of operator 100) sensors can be implanted into the brain of operator 100.

In one of the embodiments, the following is used at least as bioelectrical data of operator 100:
data on the brain's activity (electroencephalogram);
data on the electrical activity of the nervous system (parameters of electrical signal and applicable action potential at the moment of distribution along the nerve, electromyogram);
data on metabolic activity of various parts of the brain;
data on muscular activity (for example, eye movement).

For example, data on the brain's activity of operator 100 is collected with electrodes fixed on the head of operator 100.

In another example, data on motor activity of operator 100 is collected with electrodes fixed on the arms and legs of operator 100.

In another example, data on eye movement activity is collected with optical sensors (performance of multiple eye photography).

In one of the embodiments, collection of bioelectrical data of operator 100 is at least made with the following:
sensors, registering the presence of current or magnetic field, created by the above-mentioned current;
optical sensors, registering light (for example, taking images);
acoustic sensors, registering sound;
sensors, registering infrared radiation;
chemical sensors, registering modification of the domain's chemical composition.

For example, the change of functional status of operator 100 when performing a task can be registered by measuring the heart rate of operator 100 with an acoustic sensor, by increasing brain activity of operator 100 with sensors, registering electromagnetic radiation (for example, electromagnetic potential) etc.

In another example, the definition of the area of focus of operator 100 is made with optical sensors, registering data on the condition of the pupils of operator 100.

In one of the embodiments, bioelectrical data collection means (device) 110 is an external means (device), independent of other systemic means (devices) and exchanging data with standardized interface.

For example, the following can act as a collection means (device): head-pieces by various manufacturers with built-in electromagnetic sensors, a microphone and a video camera, a controller, digitizing means (device) and means (device) performing primary processing of data, collected by sensors, and means (device) transferring the collected data by cable with USB interface or by wireless interfaces, such as Wi-Fi or Bluetooth.

In one of the embodiments, bioelectrical data collection means (device) 110 is additionally designed to digitize data, received from various sensors and to translated the digitized data to the unified pre-configured form.

For example, an electroencephalogram, parameters of electric signals and an applicable action potential at the moment of its distribution along the nerve, an electromyogram, an audio recording (for example, recording of the heart rhythm of operator 100), a video recording (for example, changes in the position and dimensions of the pupils of operator 100) after the above-mentioned processing are translated into the form, described with amplitude-time dependence Aᵢ(t); at this, the information from every type of sensors can be processed independently (in this case, there will be several data channels, characterized with various amplitude-time dependencies).

In one of the embodiments, the collected bioelectrical data of operator 100 is the combination of dimensions {Aᵢ, tᵢ, p₁, p₂, . . . pₙ}, where {pⱼ} are the parameters of dimension i, which is at least described by:
amplitude-time dependence Aᵢ(t), i.e. the combination of sensor values (amplitudes), received in definite time lapses; at this, the combination of measures can be characterized with various amplitude-time dependencies for various frequency ranges A_{ω}(t) frequency-time dependence ω(t).

For example, data on the brain's activity of operator 100, represented as EEG, can be grouped in several channels and described with amplitude-time dependencies for various frequencies, for example,
Channel No. 1: Alpha rhythm (α-rhythm)—a vibration frequency range from 8 to 13 Hz. The amplitude is 5-100 microvolts, the maximal amplitude is shown with eyes closed;
Channel No. 2: Beta-rhythm (β-rhythm)—a vibration frequency range from 14 to 40 Hz. The vibration amplitude is usually up to 20 microvolts. Normally, it is poorly expressed as compared with other rhythms and mostly have the amplitude of 3-7 microvolts,
Channel No. 3: Gamma-rhythm (γ-rhythm)—a vibration frequency is over 30 Hz, sometimes reaching 100 Hz, the amplitude usually does not exceed 15 microvolts;
Channel No. 4: Delta-rhythm (δ-rhythm)—a vibration frequency varies from 1 to 4 Hz; the amplitude is within 20-200 microvolts (high-amplitude waves).

In one of the embodiments, bioelectrical data collection means (device) 110 is additionally designed to preliminarily process the collected bioelectrical data in order to eliminate artefacts (for example, in order to reduce noise) from the collected bioelectrical data.

For example, after receiving EEG, artefacts, occurring to eye movement and muscular activity of operator 100, are detected and removed:
for eye movement artefacts, the parts of EEG recording are made with individual parameters of eye movement artefacts—according to the exceedance of threshold amplitude;
for muscular artefacts, the parts of EEG recording with high-amplitude content are removed. The signal is filtered.

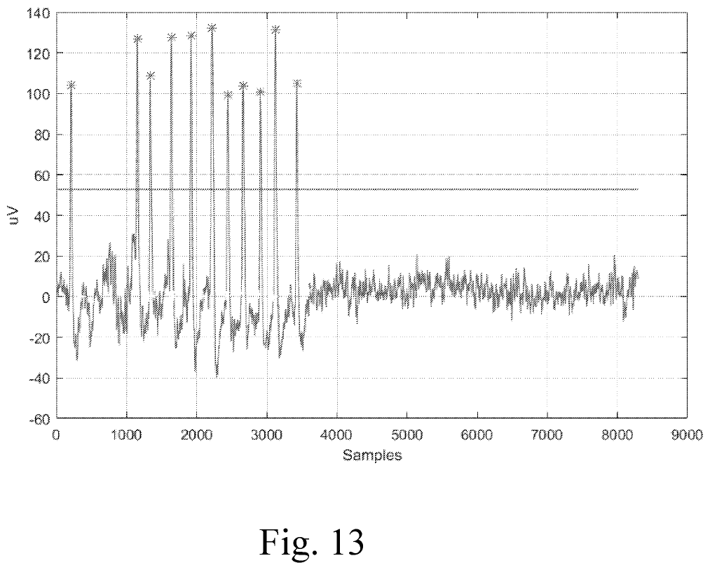

For automated removal of eye movement artefacts, 10-15 seconds EEG recording is made, during which operator 100 is instructed to blink freely several times. According to this record, an average blink amplitude and average length of blink is defined. Based on the calculated amplitude, the limit is set, the exceedance of which proves an artefact. For automated detection of artefacts, the threshold from the maximal peak at the test area with artefacts is made; the length of eye movement artefact is calculated from the peak of blink to the second cross with the signal and the isoline (FIG. 13). When the procedure of artefact removal fixes the threshold exceedance, bioelectrical data collection means (device) 110 checks, how many samples (data, which present combinations of measures describing single imaginary movements) relate to blinks and marks the current sample and the fulfilling sample, if necessary, as artefacts (in case the artefact occurred at the margin between two samples).

To remove muscular artefacts, the system takes the following parameters: frequency range and threshold amplitude. Fourier transformation is calculated for every EEG channel and amplitude values are checked within the selected frequency range. In case the amplitude is exceeded, the sample is marked as an artefact and is excluded from the following analysis. According to the values of amplitudes within the given frequency range the presence of muscular artefacts of signals is defined in real time.

Feature extraction means (device) 120 is designed to:
calculate the characteristic features for the acquired bioelectrical data of operator 100 based on feature extraction rules 121; at this, the characteristic features are parameters, describing the above-mentioned bioelectrical data with the configured accuracy (allowing to differentiate data);
transfer the calculated characteristic features to action pattern classification means (device) 130.

In one of the embodiments, the accuracy of the calculated parameters is set based on the statistical data on the described system performance used with other operators 100.

For example, bioelectrical data of operator 100 can be described by various curves fᵢ(pᵢ₁, pᵢ₂ . . . pᵢₙ) from feature extraction rules 121. The curve is selected, which described the collected bioelectrical data more accurately among all the available curves {fᵢ}. The accuracy is determined by one of the regression analysis methods. At this, the calculated parameters {pᵢ} will be the desired features of the collected bioelectrical data.

In one of the embodiments, feature extraction rules 121 are determined beforehand by any available technical method based on collected bioelectrical data from other operators 100 (for example, at the stage of development and quality analysis of the described system), or are theoretically calculated based on the existing biological models.

For example, based on the collected bioelectrical data of operator 100 and data on which operators 100 were going to perform actions, we can determine feature extraction rules 121 and the most optimal characteristics of the above-mentioned rules. For example, the following can act as such rules and characteristics:
dimensions of segments into which bioelectrical data of operator 100 will be divided before the subsequent processing;
types and parameters of curves describing bioelectrical data of operator 100;
parameters of analysis sliding window when segmenting bioelectrical data of operator 100 (for example, a sliding window step, sliding window dimensions etc.).

In one of the embodiments, feature extraction means (device) 120 is additionally designed for preliminary analysis of the acquired bioelectrical data (represented as a signal, i.e. the combination of dimensions, describe with time dependency), at which the following occurs at least:
a signal, described with amplitude-time dependency, transforms into a signal, described with amplitude-frequency dependency, and vice versa (for example, with the use of Fourier transformation);
division of the signal into several channels, i.e. the extraction of several new signals from one signal (for example, for the configured frequency ranges);
multiplication of signal of several channels into one.

In one of the embodiments, the following at least acts as the characteristic features of bioelectrical data of operator 100, which can be segmented; each segment is describe by the curve fⱼ=Fⱼ(x₁, x₂, . . . , xₙ):
the type of curve fⱼ of the segment j (i.e. which equation it can be described with);
the area under the curve fⱼ of the segment j;
the complexity of the curve fⱼ of the segment j (i.e. a numerical characteristic describing the special points of the curve);
parameters of Fourier transformation;
parameters of wavelet decomposition of the curve fⱼ of the segment j;
correlation of calculated parameters of EEG signal and other bioelectrical data of test subjects.

For example, calculation of the area under curve fⱼ of segment j (signal) can consist of three stages:
1) at the first stage, the values of the signal amplitude raise over the isoline:

$$f ( {x}_{i} ) = f ( {x}_{i} ) + \vert min ( f ) \vert$$

$$i ∋ [ 1 , N ]$$

where N is a number of points in the EEG record.
2) at the second stage, the areas under the curve fⱼ of the segment j are calculated with a trapezoidal method between the pairs of the neighboring counts:

S(xᵢ)=½(f(xᵢ₊₁)+f(xᵢ))×h
3) at the third stage, the final value for the area under the curve is calculated at the sum of the resulting values S(xᵢ) inside segment j:

$${S}_{j} = {\sum }_{i = 1}^{n} S ( {x}_{i} )$$

$$j ∋ [ 1 , k ]$$

where n is the length of the segment and k is a number of segments.

In another example, the curve fⱼ of the segment j can be calculated by counting the length of piecewise-linear approximation of the curve fⱼ. For this, Pythagoras' theorem is used to calculate the length of the gap between the neighboring counts in each pair:

$$L ( {x}_{i} ) = \sqrt{{( f ( {x}_{i + 1} ) - f ( {x}_{i} ) )}^{2} + {( {x}_{i + 1} - {x}_{i} )}^{2}}$$

$$i ∋ [ 1 , N ]$$

where n is the length of the segment.

In another example, wavelet decomposition is an integral decomposition, allowing to acquire time-and-frequency representation of function fⱼ. Basic wavelet functions allow focusing on the local features of the analyzed processes, which cannot be detected with traditional Fourier and Laplace transformations. The crucial significance is the possibility of wavelets to analyze non stationary signals with modified componential content in time or in space.

For instance, continuous wavelet transformations can be used based on various wavelet types (Morlet, Symlets etc.). The above-mentioned wavelets were selected based on the results known from the technical level, showing the efficiency of such parent wavelets in EEG analysis. In operation of feature extraction means (device) 120 Morlet and Symlets wavelets of the 4^{th} order can be used. The following scales of wavelets can be used for the above-mentioned wavelets: Morlet of the 4^{th} order with the scale of 18 Hz and 41 Hz, which correspond to 22 and 10 Hz central frequencies; Symlets of the 4^{th} order with the scale of 16 Hz and 36 Hz, which also correspond to the above-mentioned central frequencies.

In the result of continuous wavelet decomposition, we get a vector of high-dimension characteristic features. To decrease the dimension of feature space an aggregation function can be used, which us the calculation of the curve complexity, acquired after wavelet decomposition.

In another example, discrete wavelet decomposition can be used. The discrete wavelet decomposition is calculated at several stages:
1) a signal is passed through a low-frequency filter with an impulse response; we get a convolution;
2) at the same time, the signal is passed through a high-frequency filter;
3) the acquired signals can be singled in 2 times.

In the result, we get detailing coefficients (after the high-frequency filter) and approximation coefficients (after the low-frequency filter). The used filters are interconnected and are called quadrature mirror filters (QMFs). Stages 1-3 can be repeated several times to increase frequency resolution. The process of acquiring coefficients for discrete wavelet decomposition can be represented as a tree named filter bank. The elements of this tree are subspaces with various time-and-frequency localizations.

In another example, several approaches can be used for the application of 5^{th} level coefficients of discrete wavelet decomposition, acting as characteristic features of EEG signals when imaging movement for the further submit to the classifier committee.
coefficients (parameters) of discrete wavelet decomposition of the 4^{th} and 5^{th} level can be transmitted directly to the classifier. In this case, wavelet decomposition acts as the first level classification with deep learning, the level of decreasing the dimension of input data space. The input of the decomposition is EEG raw signal (a signal, corresponding to each following sample, is a vector, the length of which depends on the length of the sample and the sampling frequency of the registering equipment), and the output is the details of the 4^{th} and 5^{th} levels, consisting of 27 and 18 values correspondingly, calculated with wavelet decomposition (in general, the number of values depends on the length of the input signal).
coefficients (parameters) of discrete wavelet decomposition of the 4^{th} and 5^{th} level can be used to restore the approximations of signal details in various frequency ranges, corresponding to every decomposition level. Next, the length of the envelope and the area of segments under the curve are calculated in the sliding windows of the analysis. These features are calculated for the approximations of details of the 4^{th} and 5^{th} level for every following sample (a single-trial approach) with first level classifiers.

In general, the analysis of features, calculated with wavelet decompositions, shows a higher information value of the signal components in the observed range of 0.5-30 Hz. As of that, coefficients of 20-25 Hz band of wavelet decompositions proved to be more informative than coefficients of 6-12 Hz band. Moreover, the “complexity of curve” meta feature, calculated for the approximations of details of wavelet decomposition of every following sample, proved to be more informative for committee of classifiers, than the “area under the segments of the approximation curve” feature, which may prove the higher importance of information on high-frequency details of the signals as compared with information on its trend.

The use of in-line wavelet transformation to the unprocessed EEG signal has potential due to several reasons, among which is the possibility to extract signal details in various scales and various frequency bands, as well as the possibility to considerably decrease the dimensions of input data for the subsequent classification by selecting relevant coefficients of only few decomposition levels. In this case, such decomposition can be considered as the variant of convolution in the first layers of a deep neural network, detecting key features and dropping excessive data.

The above-mentioned methods to calculate characteristic features do not have severe requirements of computational powers and have low calculation time.

The presented system suggests a dynamic configuration of a wavelet decomposition step and individual approach to the definition of central frequencies of EEG signals in various ranges during wavelet decompositions of every operator 100.

In one of the embodiments, feature extraction means (device) 120 is additionally designed to simultaneously account for the features of two-level committee of local classifiers, in which the lower level contains at least two artificial neural networks and at least two support vector machines, and the upper level contains an artificial neural network, which unites classification results of the lower level.

Action pattern classification means (device) 130 is designed to:
generate action patterns based on the acquired characteristic features using action pattern classification model 131;
identify generated action patterns; at that, when identifying generated action patterns have at least one corresponding pattern from action pattern base 132;
transfer of identified generated action patterns to control command generation means (device) 150.

In one of the embodiments, action pattern classification model 131 is a combination of action pattern rules based on at least one action pattern from action pattern base 132.

It is impossible to describe EEG signal, corresponding to a movement, due to the variability of EEG. We can also say that an equation is a regression model of the signal with the minimal error from those included into the model set. For example, an EEG segment, registered in the range Δt=1.17-1.77c, can be characterized by the curve equation A(t)=Σ0.23×sin(4.25×t−0.12)e^{-4.12t}^{²}, the combination of the mentioned curve characterizes brain activity, related to the operation of bending the pointer finger.

In one of the embodiments, a pattern classification model is an artificial neural network and is preliminarily generated with machine learning methods.

For example, to recognize imaginary movements on EEG, patterns of actions are configured in advance, which are based on support vector machines and artificial neural networks. The given approaches are effective classification methods, including application with multichannel EEG signals.

The applied support vector machines method belongs to linear classification methods. The essence of the method is the separation of the sample into classes with optimal separating hyperplane, the equation of which in the general case is as follows:

$$f ( x ) = ( ω , ( x ) ) + b$$

$$where$$

$$ω = {\sum }_{i = 1}^{N} {λ}_{i} {y}_{i} ( {x}_{i} )$$

coefficients λᵢ depend on yᵢ (vectors of class marks) and on the value of scalar products (xᵢ, xⱼ). Thus, to find the decision function it is necessary to know the values of scalar products. Data transformations are defined according to the kernel function:

K(x,y)=(x,y)

Based on the study results on the selection of the preferred SVM type for EEG signal classification, Gaussian radial basic function SVM-RBF SVM is applied as a kernel function:

K(xᵢ,xⱼ)=e^{-γx}^{ᵢ}^{-x}^{ⱼ}²

for g>0.

The above mentioned artificial neural networks (ANNs) are based on the principles of distributional, non-linear and parallel data processing with learning. In the above-mentioned example, ANNs are implemented in the form of a multi-layer perceptron, consisting of three layers: two discrete layers and one output layer. A sigmoid function is used as a function to activate in discrete layers

$$out = \frac{1}{1 + {e}^{- α Y}}$$

where α—a slope parameter of the sigmoid function, and a linear function in the output layer.

For the purpose of simultaneous accounting of several types of features, a 2-level committee of local classifiers is used, the lower level of which consists of 2 ANNs and 2 support vector machines. The upper level consists of an ANN, which unites the classification results of the lower level.

The following are used as features:
area under the curve,
the curve complexity,
discrete and continuous wavelet decomposition coefficients.

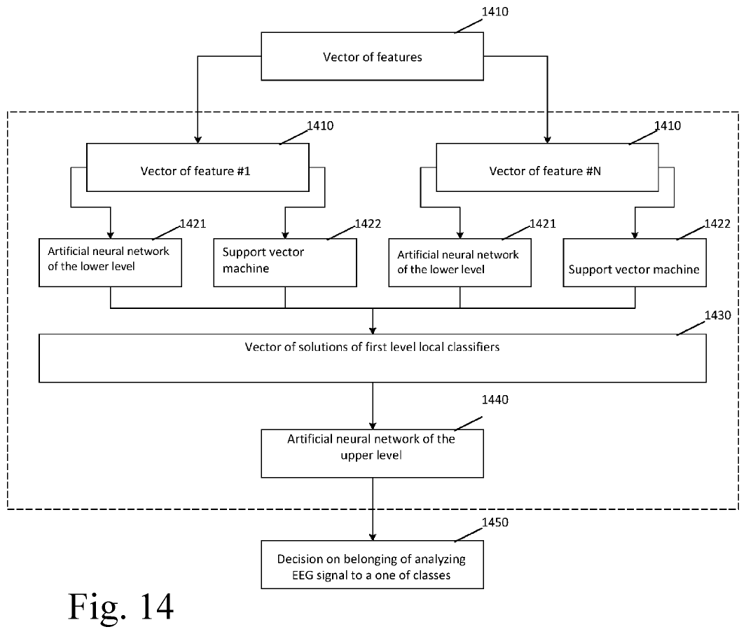

Lower-level classifiers input the features of various types and decide on classification of the given EEG signal. These decisions are summarized into a vector and are input to the ANN of the upper level, which performs the final classification, i.e. relates the analyzed EEG signal to one of the classes (FIG. 14). Thus, there is a possibility to select the best features for classification.

The upper-level ANN is trained on a dataset, including the solutions from the lower-level local classifiers. The trained upper-level ANN defines the importance of the solutions of every lower-level classifier and performs the selection of the best solution.

Due to the implemented structure, the described system can be individually built in for operator 100, allowing the selection of the most relevant features, whereas the committee of classifiers is easily scaled, including new lower-level classifiers.

In one of the embodiments, the identification of action patterns involves at least the following:
determination of which of action patterns from action pattern base 132 is more similar to the generated pattern;
definition of the parameters of the generated pattern based on the acquired features and parameter of the found pattern and action pattern base 132.

In one of the embodiments, the action pattern is at least characterized by:
action type;
parameters, describing the action of the given type.

In one of the embodiments, action pattern classification means (device) 130 is additionally designed to transfer the acquired characteristic features to overtraining means (device) 140 to overtrain action pattern classification models 131.

Overtraining means (device) 140 is designed to overtrain action pattern classification model 131 so that the following results at least:
a number of errors, made by operator 100 during control command generation would be below the set threshold;
the preconfigured extracted features would be sufficient to generate the configured action pattern.

Control command generation means (device) 150 is designed to:
generate at least one control command by external control means (device) 151 based on the acquired action patterns;
transfer the generated command to external means (device) 151.

In one of the embodiments, generation of control commands at least contains a stage, at which:
an acquired action pattern has at least one corresponding control command by external means (device) 151;
parameters of the corresponding control commands are calculated based on the parameters of the acquired action pattern and the peculiarities of the operation of external means (device) 151.

For example, the acquired action pattern of bending the pointer finger phalanx corresponds to the electromotor control command #r2f2 on the right arm prosthesis of operator 100. The parameter of the mentioned pattern of action performance speed and action performance force corresponds to 1 m/s and 2N correspondingly, which after the transfer into control commands by the described electromotor means the voltage and current rate for electromotor of 2.4V and 0.03 A.

In another example, the pattern of the action “moving the mouse cursor” is converted into the data on a relative mouse cursor shift on the display for the configured values (Dc, Ay).

In one of the embodiments, the following at least acts as external means (device) 151:
a computer (or any other calculation means (device), a tablet, a phone etc.), for which the described system acts as an information input means (device) (for example, a game pad, a pointing means (device) etc.);
a biomechanical prosthesis;
a mechanical mobility means (device) (for example, a mobility scooter);
a communication and assistance means (device)—a robot assistant;
program keyboard control for communication;
heating appliances (blankets, gloves, socks);
site management;
speech generation means (device).

In one of the embodiments, external means (device) 151 is a smart home component, i.e. a component of the system of household appliances, which are able to make actions and solve certain routine tasks without human participation.

For example, operator 100 can use the described system to control smart home elements, particularly to manage air conditioning and lighting modes in the room, control the operation of TV and home theater.

In another example, operator 100 (for instance, a person with previous stroke) can use the described system to control the bed configuration (for instance, to control the slope of the bed, of the head rests, to call medical assistants etc.).

In another example, operator 100 (for instance, an amputee) can use the described system to control the bioelectrical arm prothesis. The system determines the desired actions of operator 100 (for instance, to bend fingers in order to catch an item), generates these actions into the corresponding commands and transfers these commands to the prosthesis, which performs the desired action with the built-in electromotors.

In one of the embodiments, external means (device) 151 additionally has the functions, providing feedback with the described system: for this:
external means (device) 151 monitors the performance of control command (by calculating the performance parameters for the control command);
the performance parameters for the control command are compared with the parameters of reference control commands of external means (device) 151;
in case the parameters of the monitored control commands differ from the parameters of the reference control commands (the values exceed), overtraining means (device) 140 is requested to overtrain pattern classification model;
overtraining means (device) 140 overtrains action pattern classification model 131 so that next time a control command, generated with action pattern, would satisfy the reference control command.

For example, controlling the mouse cursor leads to the situation, when the cursor starts to shift to the left, though the task, performed by 100, requires holding the cursor straight, i.e. an excessive horizontal shift occurs when generating the control command. This information is submitted to overtraining means (device) 140, which leads to the decrease of the shift.

Thus, individual calibration of the described system occurs considering the particular operator 100.

In one of the embodiments, before operator 100 starts using the described system to control external means (device)s 151 the described system is calibrated. For this, the following occurs at least:
operator 100 performs actions, for which reference commands exist, which helps to configure correct parameters for the commands;
command performance artefacts are determined.

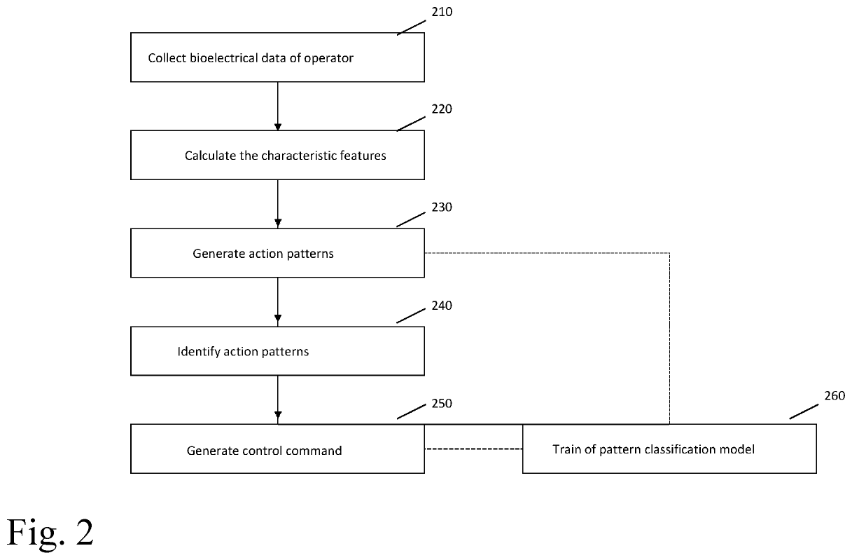

FIG. 2 is a flowchart of the method of generating control commands based on Operator's bioelectrical data.

A flowchart of the method of generating control commands based on bioelectrical data of the operator consists of stage 210, at which Operator's bioelectrical data is collected, stage 220, at which the characteristic features are calculated, stage 230, at which action patterns are generated, stage 240, at which action patterns are identified, stage 250, at which control commands are generated, stage 260, at which the pattern classification model is trained.

At stage 210, bioelectrical data collection means (device) 110 is used to collect bioelectrical data of operator 100.

At stage 220, feature extraction means (device) 120 is used to calculate the characteristic features of bioelectrical data of operator 100 collected at stage 210 based feature extraction rules 121; at that, the characteristic features are parameters describing the above-mentioned bioelectrical data with the configured accuracy.

At stage 230, action pattern classification means (device) 130 is used to generate action patterns based on the characteristic features calculated at stage 220 using action pattern classification model 131.

At stage 240, action pattern classification means (device) 130 is used to identify action patterns, generated at stage 230, whereas during identification, the generated action patterns have at least one corresponding pattern from action pattern base 132.

At stage 250, control command generation means (device) 150 is used to generate at least one control command for external means (device) 151 based on action patterns identified at stage 240.

At stage 260, overtraining means (device) 140 is used to overtrain pattern classification models so that
a number of errors, made by 100 when generating commands would be below the configured threshold;
the pre-configured extracted features would be sufficient to generate the configured action pattern.

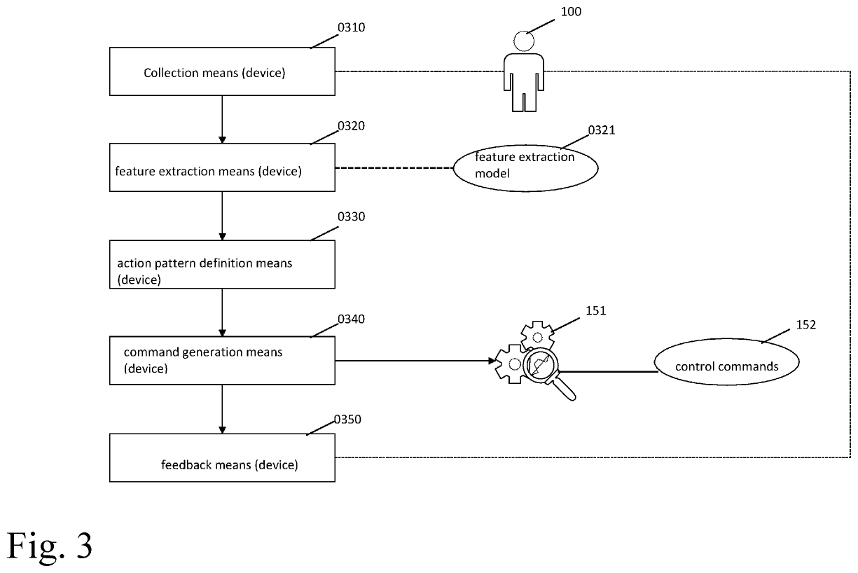

FIG. 3 is an example of the flowchart of the system of generating control commands based on operator's bioelectrical data. FIG. 3 shows an example of a structural configuration for the control command formation system based on Operator's bioelectrical data.

A flowchart of the system of generating control commands based on operator's bioelectrical data contains collection means (device) 0310, feature extraction means (device) 0320, action pattern definition means (device) 0330, command generation means (device) 0340, feedback means (device) 0350.

Collection means (device) 0310 is designed to collect bioelectrical data of operator 100 and to transfer the collected data to feature extraction means (device) 0320, while the following acts as bioelectrical data:
electroencephalogram of operator 100, where an electroencephalogram is a set of activity signals of the nervous system of operator 100, characterized with the signal registration time and the signal amplitude (further, an EEG signal),
electromyogram of operator 100, where an electromyogram is a set of activity signals of the muscular system of operator 100, characterized with the signal registration time and the signal amplitude (further, an EMG signal).

In one of the embodiments of the system, collection means (device) 0310 is additionally designed to extract at least two samples from the collected bioelectrical data, where each sample is a set of data describing a single image of the movement of operator 100.

In one of the embodiments of the system, after bioelectrical data is collected, the data is analyzed and converted, for which the following is made at least:
high and low frequency filters are used,
network noise is removed, using at least band elimination and band-pass filters,
time stamp synchronization is made,
oculographic artefacts are removed,
myographic artefacts are removed,
a filtered EEG signal is used;
an EEG signal is transformed for mean, weighed mean composition, current source density, topographies of independent components.

Feature extraction means (device) 0320 is designed to extract the characteristic features from the collected bioelectrical data with the following:
trained feature extraction model 0321, formed on the basis of machine learning methods,
a set of feature extraction rules;
at that, the characteristic features include:
spectral characteristics,
time characteristics,
wavelet decomposition characteristics;
spatiotemporal characteristics,
a combination of characteristics of bioelectrical activity of various genesis;
and to transfer the extracted characteristics to action pattern definition means (device) 0330.

In one of the embodiments of the system, the action pattern is defined with a two-level committee of local classifiers, in which the lower level contains a combination of at least one classifier based on support vector machine and at least one artificial neural network, and the upper level contains at least one artificial neural network.

In one of the embodiments of the system, the action pattern is defined with a two-level committee of local classifiers, in which the lower level contains at least two classifiers based on discriminant data mining or two support vector machines, and the upper level contains at least one artificial neural network. In one of the embodiments of the system, an artificial neural network of the upper level of the committee of local classifiers is trained on the combination of data, containing the solutions from every local classifier of the lower level.

Action pattern definition means (device) 0330 is designed to define an action pattern under the extracted characteristic features with artificial intelligence methods and to transfer a certain action pattern to command generation means (device) 0340, whereas an action pattern is a numerical value to characterize the probability that the collected bioelectrical data of operator 100 belongs to the configured imaginary action of operator 100.

Command generation means (device) 0340 is designed to generate control command 152 with external means (device) 151 based on a certain action pattern.

Feedback means (device) 0350 is designed to make the following based on a certain action pattern:
to generated an image of the above-mentioned action to display to operator 100;
to imitate the above-mentioned action with external means (device) 151;
to generated an image of the parameters of bioelectrical data, related to a certain action pattern;
to perform other actions, related to the above-mentioned action.

For example, imagining a hand clap, the user activates light on (in a smart home), i.e. the clapping action results in the performance of the action of a different type (not related to hands clapping or occurring due to a slapping sound)—turning on lights.

Additionally, the system of generating control commands based on Operator's bioelectrical data can contain visualization tools for operator's action, when each imaginary action is visualized for operator during recognition.

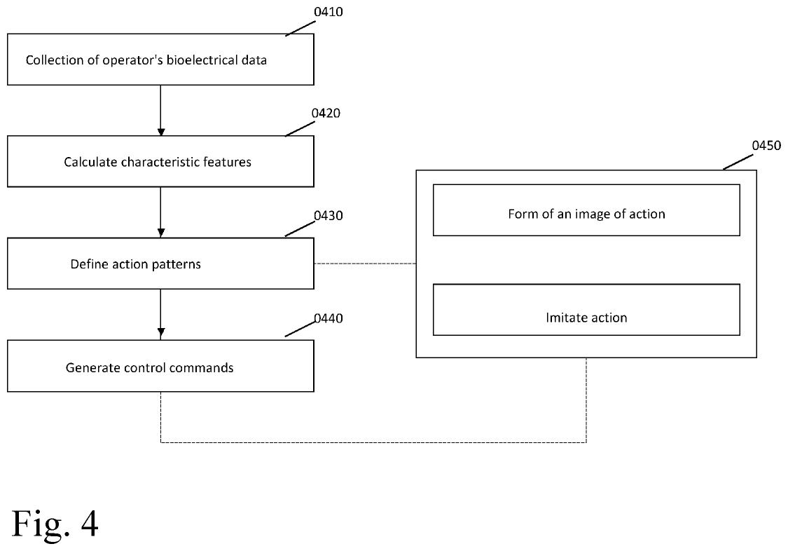

FIG. 4 is an example of the flowchart of the method of generating control commands based on Operator's bioelectrical data.

A flowchart of the method of generating control commands based on Operator's bioelectrical data contains 0410, at which bioelectrical data of operator 100 are collected, stage 0420, at which the characteristic features are calculated, stage 0430, at which action patterns are defined, stage 0440, at which control commands are generated.

The mentioned stages 0410-0440 are implemented with the means (device)s of the system shown in FIG. 3.

At stage 0410, collection means (device) 0310 is used to collect bioelectrical data of operator 100; at that, the following acts as bioelectrical data:
an electroencephalogram of operator 100, where an electroencephalogram is a set of activity signals of the nervous system of operator 100; the set is characterized with the signal registration time and the signal amplitude (further, an EEG signal),
an electromyogram of operator 100, where an electromyogram is a set of activity signals of the muscular system of operator 100, characterized with the signal registration time and the signal amplitude (further, an EMG signal).

In one of the embodiments of the method, at least two samples are extracted from the collected bioelectrical data, and the subsequent analysis, including stages 0420-0440 is made at least for one extracted sample; this is a set of data describing a single image of the movement of operator 100.

In one of the embodiments of the method, after the bioelectrical data is collected, the analysis and transformation of the collected data is made, for which the following is made at least:
high and low frequency filters are used,
network noise is removed, using at least band elimination and band-pass filters,
time stamp synchronization is made,
oculographic artefacts are removed,
myographic artefacts are removed,
a filtered EEG signal is used;
an EEG signal is transformed for mean, weighed mean composition, current source density, topographies of independent components.

At stage 0420, feature extraction means (device) 0320 is used to extract the characteristic features from the collected bioelectrical data using the following:
trained feature extraction model 0321, formed on the basis of machine learning methods,
a set of feature extraction rules;
at that, the characteristic features include:
spectral characteristics,
time characteristics,
wavelet decomposition characteristics;
spatiotemporal characteristics,
a combination of characteristics of bioelectrical activity of various genesis.

In one of the embodiments of the methods, the action pattern is defined with a two-level committee of local classifiers, in which the lower level contains a combination of at least one classifier based on support vector machine and at least one artificial neural network, and the upper level contains at least one artificial neural network.

In one of the embodiments of the methods, an artificial neural network of the upper level of committee of local classifiers is trained on a dataset, containing the solutions for every local classifiers of the lower level.

At stage 0430, action pattern definition means (device) 0330 is used to define an action pattern under the extracted characteristic features using artificial intelligence method; the action pattern is a numerical value, which characterizes the possibility of whether the collected bioelectrical data of operator 100 belong to the configured imaginary action of operator 100.

At stage 0440, command generation means (device) 0340 is used to generate control command 152 with external means (device) 151 based on a specific action pattern.

At stage 0450, feedback means (device) 0350 is additionally used to do the following on the basis of the defined action pattern:
form of an image of the mentioned action to display to operator 100;
imitate the mentioned action with external means (device)s 151;
form the visual image of parameters of bioelectrical data, related to the specific action pattern;
perform actions of a different nature, related to the mentioned action.

In an enlarged sense (having a wider functionality), the above-mentioned method of generating control commands based on bioelectrical data of operator 100 can include the following stages:

At stage 0410 bioelectrical data of operator 100 is collected.

In a particular embodiment of the method, an electroencephalogram of operator 100 acts as bioelectrical data of operator 100, where an electroencephalogram is a set of activity signals of the operator's nervous system; the set is characterized with the signal registration time and the signal amplitude (further, an EEG signal).

In another particular embodiment of the method, at least two samples are preliminarily extracted, and the subsequent analysis, including stages 0420-0440 is made at least for one extracted sample, whereas every sample is a set of data describing a single image of the movement.

At stage 0420, characteristic features from the collected bioelectrical data are extracted.

In a particular embodiment of the method, the characteristic features of operator 100 are extracted with trained feature extraction model 0321, generated on the basis of machine learning method.

In another particular embodiment of the method, the following is at least extracted:
time features,
frequency features.

In another particular embodiment of the method, the characteristic features are extracted with wavelet decomposition.

In another particular embodiment of the method, the following at least act as characteristic features:
area under the curve of EEG-signal,
complexity of the curve of EEG-signal,
wavelet decomposition coefficients of the curve of EEG-signal.

In another particular embodiment of the method, the following is used at least to calculate the characteristic features:
an algorithm to calculate the area under the curve of EEG-signal,
an algorithm to calculate complexity of the curve of EEG-signal in sliding window,
an algorithm to calculate wavelet decomposition of the curve of EEG-signal,
an algorithm to calculate cepstral coefficients.

At stage 0430, at least one action pattern is defined with the extracted characteristic features.

In a particular embodiment of the method, the action pattern is a numerical value, which characterizes the possibility of whether the collected bioelectrical data of operator 100 belongs to the configure imagine action of operator 100.

In another particular embodiment of the method, the action pattern is defined at least with the following:
support vector machine;
artificial neural network.

In another particular embodiment of the method, the action pattern is defined with a two-level committee of local classifiers, in which the lower level contains a combination of at least one classifier based on support vector machine and at least one artificial neural network, and the upper level contains at least one artificial neural network.

In another particular embodiment of the method, an artificial neural network is trained on the combination of data, containing the solutions of every item of the set of local lower-level classifiers.

At stage 0440, at least one control command for an external means (device) is generated based on at least one defined action pattern.

In a particular embodiment of the method, after the bioelectrical data is collected, the analysis and transformation of the collected data is additionally made, for which the following is made at least:
high and low frequency filters are used,
network noise is removed, using at least band elimination and band-pass filters,
time stamp synchronization is made,
oculographic artefacts are removed,
myographic artefacts are removed,
a filtered EEG signal is used;
an EEG signal is transformed for mean, weighed mean composition, current source density, topographies of independent components.

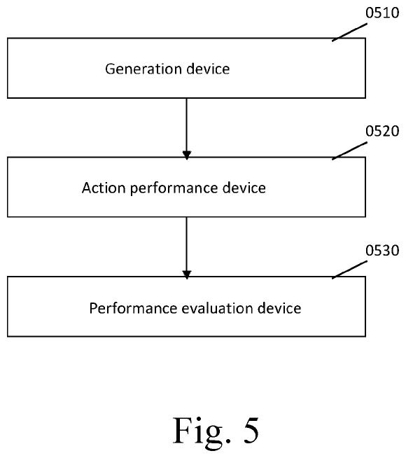

FIG. 5 is an example of the flowchart of task execution performance evaluation system based on bioelectrical data of operator.

A flowchart of task execution performance evaluation system based on bioelectrical data of operator consists of generation means (device) 0510, action performance means (device) 0520, performance evaluation means (device) 0530.

Generation means (device) 0510 is designed to generate the following under the preconfigured rules:
a virtual domain, including at least one virtual object; at that, the state of the virtual object is characterized at least by the following:
   Position in the virtual domain,
   Dimensions,
   Color,
   Interaction rules for the virtual domain,
   State change rules depending on actions of operator 100 in the virtual domain;
A task for operator 100 to perform at least one action related to at least one virtual object.

In one of the embodiments of the system, the virtual domain, the virtual objects in the virtual domain and the actions, performed by operator 100 in the virtual domain, are additionally visualized.

In one of the embodiments of the system, the task includes the change of the state of at least one virtual object with at least one action made by operator 100.

In one of the embodiments of the system, to perform the task, the change of the state of the virtual object must be performed by operator 100 at least:
for the configured time,
with the configured number of tries.

In one of the embodiments of the system, the preconfigured rules for task formation include at least one control command, which must be generated based on bioelectrical data of operator 100

Action performance means (device) 0520 is designed to perform at least one action of operator 100 in the virtual domain based on the generated control command.

Performance evaluation means (device) 0530 is designed to:
evaluate the performance of the action; the performance of the action is a numerical value, characterizing the similarity of the state of the virtual object after the operator 100 performed an action at the virtual object, with the expected state of the mentioned virtual object in case the action was accurately performed by operator 100;
evaluate the task execution performance based on the action performance evaluation acquired by the same means (device) 0530; at that, the task execution performance is a numerical value, characterizing the number of errors, made by operator 100 during the performance of the action at the virtual object; an error is the performance of the action at the virtual object below the configured performance.

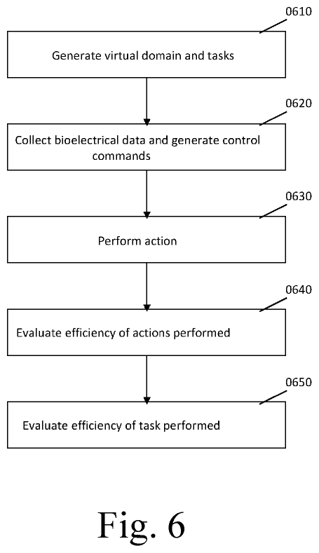

FIG. 6 is an example of the flowchart of the task execution performance evaluation method based on bioelectrical data of operator.

A flowchart of the task execution performance evaluation method based bioelectrical data of operator 100 contains stage 0610, at which the virtual domain and tasks are generated, stage 0620, at which bioelectrical data of operator 100 generate control commands, stage 0630, at which actions are performed, stage 0640, at which the action performance is evaluated, and stage 0650, at which the task performance is evaluated.

The above-mentioned stages 0610-0650 are implemented with the method from the system, shown in FIG. 5.

At stage 0610 generation means (device) 0510 is used to generate the following based on the preconfigured rules:
a virtual domain, including at least one virtual object; at that, the state of the virtual object is characterized at least by the following:
   Position in the virtual domain,
   Dimensions,
   Color,
   Interaction rules for the virtual domain,
   State change rules depending on actions by operator 100 in the virtual domain;
A task for operator 100 to perform at least one action related to at least one virtual object.

In a particular embodiment of the method, the virtual domain, the virtual objects in the virtual domain and the actions, performed by operator 100 in the virtual domain, are additionally visualized.

In another embodiment of the method, the task includes the change of state of at least one virtual object by at least one action by operator 100.

In another particular embodiment of the method, to perform the task, the change of the state of the virtual object must be performed by operator 100 at least:
for the configured time,
with the configured number of tries.

In another particular embodiment of the method, the preconfigured task generation rules include at least one control command, which must be generated based on bioelectrical data of operator 100.

At stage 0620 with stages 0410-0440 of the method of generating control commands based on bioelectrical data of operator 100 bioelectrical data of operator 100 are collected and at least one control command is generated based on the collected bioelectrical data of operator 100.

At stage 0630 action performance means (device) 0520 is used to perform at least one action by operator 100 based on generated control command.

At stage 0640 performance evaluation means (device) 0530 is used to evaluate the performance of the action; the performance of the action is a numerical value, characterizing the similarity of the state of the virtual object after the Operator performed an action at the virtual object, with the expected state of the mentioned virtual object in case the action was accurately performed by operator 100.

At stage 0650 performance evaluation means (device) 0530 is used to evaluate the task execution performance; the task execution performance is a numerical value, characterizing the number of errors, made by operator 100 during the performance of the action at the virtual object: the error is the performance of the action at the virtual object below the configured performance.

In an enlarged sense (having a wider functionality), the above-mentioned method of generating control commands based on bioelectrical data of operator 100 can include the following stages:

At stage 0610 generation means (device) 0510 is used to generate the following based on the preconfigured rules:
A virtual domain, including at least one virtual object; at that, the state of the virtual object is characterized at least by the following;
A task for operator 100 to perform at least one action related to at least one virtual object.

In a particular embodiment of the method, the virtual domain, the virtual objects in the virtual domain and the actions, performed by operator 100 in the virtual domain, are additionally visualized.

In a particular embodiment of the method, the state of the virtual object is characterized at least by the following:
position in the virtual domain;
dimensions;
color;
interaction rules for the virtual domain;
state change rules depending on action by operator 100 in the virtual domain.

In another embodiment of the method, the task includes the change of state of at least one virtual object by at least one action by operator 100.

At stage 0610 generation means (device) 0510 is used to generate the following based on the preconfigured rules:
A virtual domain, including at least one virtual object; at that, the state of the virtual object is characterized at least by the following;
A task for operator 100 to perform at least one action related to at least one virtual object.

In another embodiment of the method, the change of state of the virtual object must be performed by operator 100 at least:
for the configured time,
with the configured number of tries.

In another particular embodiment of the method, the preconfigured task generation rules include at least one control command, which must be generated based on bioelectrical data of operator 100.

At stage 0620, means (device) 0310-0340 are used to collect bioelectrical data of operator 100 and generate at least one control command based on the collected bioelectrical data of operator 100.

At stage 0630, action performance means (device) 0520 is used to perform at least one action by B operator 100 based on the generated control command.

At stage 0640, performance evaluation means (device) 0530 is used to evaluate the action performance.

In another particular embodiment of the method, the performance of the action is a numerical value, characterizing the similarity of the state of the virtual object after the Operator performed an action at the virtual object, with the expected state of the mentioned virtual object in case the action was accurately performed by operator 100.

At stage 0650, means (device) 0530 is used to evaluate the task performance efficiency based on the action performance.

In another particular embodiment of the method, the task execution performance is a numerical value, characterizing the number of errors, made by the Operator during the performance of the action at the virtual object; an error is the performance of the action at the virtual object below the configured performance.

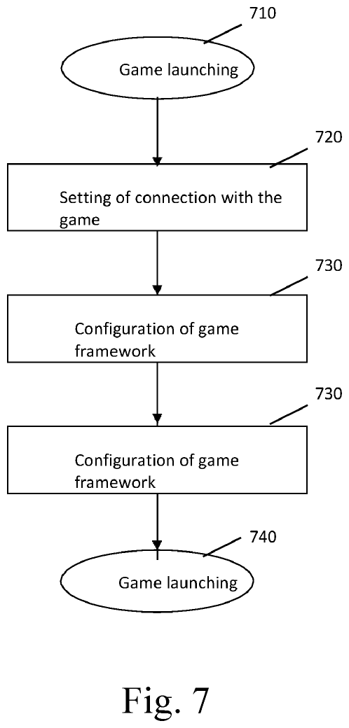

FIG. 7 is an example of the general workflow of the visual game framework with the use of the system of generating control commands based on bioelectrical data of operator.

A game form of after-care based on the system of generating control commands based on the bioelectrical data of the operator (further, a brain-computer interface, BCI) uses training of operator 100 (further, a patient) by neurofeedback. This approach focuses on the stimulation of the brain flexibility and restorative processes in the central nervous system of patient 100. The main condition for its successful application is a high motivation of patient 100. To satisfy this demand, game framework (including virtual game framework) can be used, which is controlled by a brain-computer interface platform. Thus, the character's actions in the game are controlled by the motor commands from the brain of patient 100. It gives patient 100 a presentation on the efficiency of their efforts and visualizes the improvement of motor function, especially when the performance of real movements is impossible for the patient. It gives a powerful positive effort and increases the efficiency of after-care procedures. Additionally, BCI after-care based on motor imagination in a game form does not require physical exercises, when active therapeutic physical training is not yet permitted for the patient due to the symptoms of their general condition.

The direct operation with the system of generating control commands based on bioelectrical data of the operator can be presented in the form of a game, in which the character of the virtual domain, controlled by the patient by making certain intelligent actions (imaginary movements) must for example gather fruit, growing on trees. The description of the action (gathering fruit) is focused on the development of the patient's grabbing movements.

The above-mentioned procedure is controlled with a special software. Preliminarily, the above-mentioned software allows for selecting the types of recognizable movements, as well as the sequence, in which one must imagine them.

Additionally, the above-mentioned software allows configuring how many fruits will be on the trees for every hand/arm, as well as how many correct recognitions are necessary to pick fruit, as well as the number of tries to pick fruit. You can also configure time for the game session.

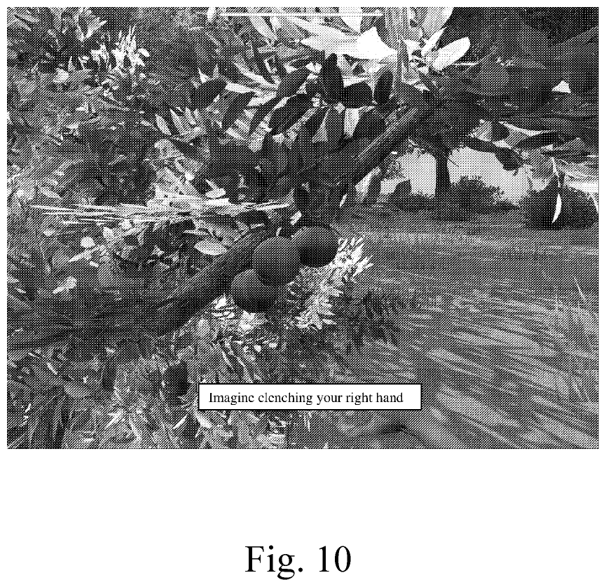

During the game (an example; does not affect the technical solution), a character moves in the garden between the trees. When the character approaches a tree, the interaction with operator 100 begins (FIG. 10).

In the lower part of the screen, the strip shows the count of tries to pick fruit from trees. In the upper part of the screen, the countdown to the game end is shown. Instructions for the patient are also given.

During the interaction, the patient must perform imaginary movements in the rhythm, set by the fruit blinking and the audio signal. In case of the correct recognition of the imaginary movement, the hand of the character approaches the fruit and picks it.

If the patient did not have enough time to pick all the fruit for the configured number of tries, the character starts approaching another tree. If the patient pick all the apples before wasting all the tries, the character also approaches another tree.

Game module algorithms:

The main stages of interaction of the classifier and the game are show in FIG. 7. After the game is launched from the user interface, the main software sets the connection with the game for data exchange. Next, it is necessary to configure the game framework in the game properties window.

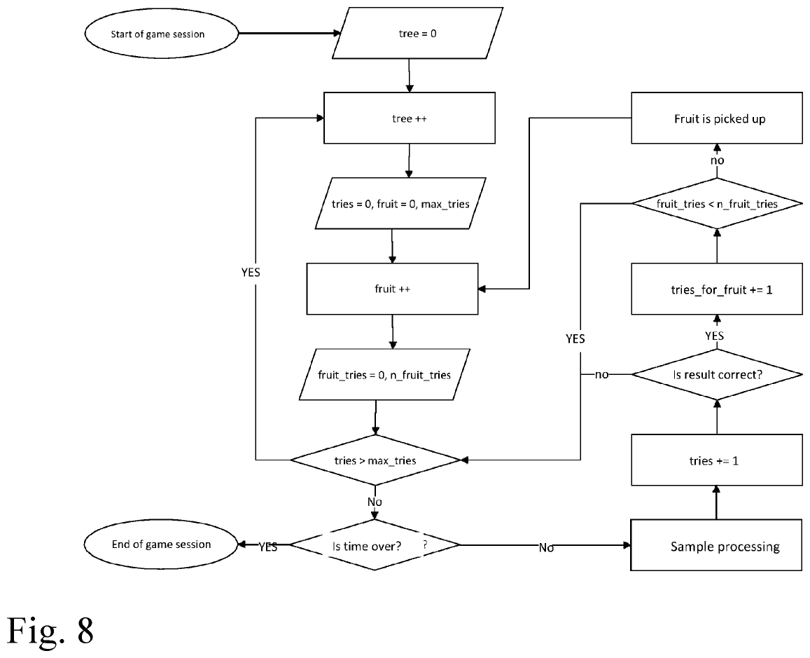

After the configuration is over, the main game session starts, in which the character moves from one tree to another and tries to pick fruit. The game operation algorithm is shown in FIG. 8. At the start of the game the character approaches the first tree, and the count of tries for one tree, which is calculated as the product of the number of fruits on the tree and the number of tries for one fruit. Each try is given a certain time, corresponding to the length of the try.

In case of correct recognition, the character's hand approaches the fruit, if the number of the required correct recognitions to pick the fruit is not reached yet, and picks it in any case.

If the patient did not have enough time to pick all the fruit for the configured number of tries, the character starts approaching another tree. If the patient pick all the apples before wasting all the tries, the character also approaches another tree.

When the time is over, the game session is over, the game closes, and the user interface shows the results of the training.

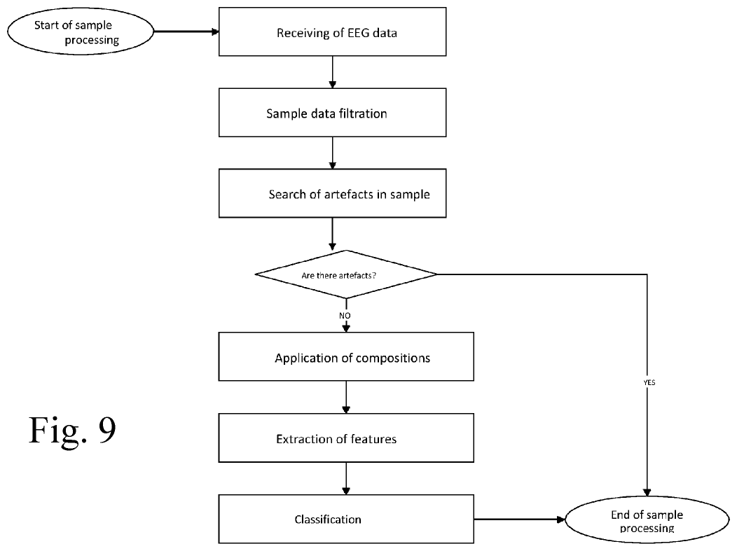

Sample processing occurs in several stages (FIG. 9). First, when the signal on the start of the sample appears, the corresponding data sample is extracted, which corresponds to the data sample from the flow accepting data. Next, data is filtered; in case the sample contains artefacts, the sample is marked as artifactual, and processing stops. If there are no artefacts, one of the compositions is used, the features are extracted and classification is performed. At the output, you either get a mark corresponding to a movement, or a mark meaning that the sample has artefacts and it is not suitable for classification.

According to the game results, all the statistical data on the number of the acquired motor commands, successfully picked fruit, as well as original data are saved in special files. The dynamics of the user's results in the game is an important marker, which means the restoration of functions to plan and perform movements, especially in case if the performance of real movement is not yet accomplished by the user.

FIG. 13 is an example of EEG with artefacts.

The operation of the system of generating control commands based on bioelectrical data of operator in real time has a number of peculiarities and limitation, the main of which is the limitation of operating time of algorithms. The implementation of the system of generating control commands based on bioelectrical data of operator, applicable in practice, is only possible in case the methods and algorithms are used, which satisfy the given limitation. Thus, when selecting the signal filtration methods, we can refuse digital filters with finite-impulse response, the use of which allows receiving a signal of a higher quality, but calculations take too much time.

To synchronize stimulations and records, EEG can use a hardware synchronization unit. The mentioned unit can be used as follows: an audio stimulation from the computer is given into the headphones of operator 100 and into the hardware synchronization unit at the same time; when crossing the threshold value, the unit sends a mark to the dedicated poly-channel of the electroencephalograph (AEIX) through the infrared port.

During the data separation, first synchro-impulses in the AEGC channel are found and time marks are calculated, which correspond to the peaks. Based on the acquired time marks, the signal is separated into samples, to which marks are assigned according to the test protocol.

The EEG signal registration system includes the configurable filtration module for input of EEG data with the use of a special bandpass and low and high frequency filter depression. A set of high frequency filters (0.016 Hz, 0.032 Hz, 0.53 Hz, 1.6 Hz, 5.3 Hz) and low frequency filters (15 Hz, 30 Hz, 50 Hz) is implemented. During filtration, to form a bandpass and network noise depression of 50 (60) Hz, continuous impulse response filters are used, which simulate RC chains more accurately and which are widely used in clinical paper electroencephalographs. To form a bandpass, a high frequency filter and a low frequency filter are implemented.

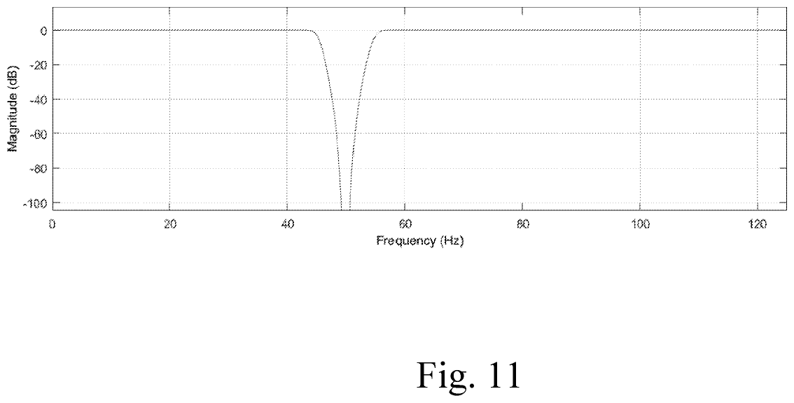

To depress network noise, 50 (60) Hz band elimination filters are used (FIG. 11). Additional 100 (120) Hz band elimination filters can also be included to depress the second harmonic of network noise. All the band elimination filters of the 12^{th} order with the reject band: 45-55, 40-60 or 35-65 Hz, depending on the configured parameters.

Additionally, the EEG registration system can implement automated detection modules for artefacts in on-line mode: the detection of eye movement (FIG. 13) and muscular artefacts based on the 2 possible procedures:
1. Automated calculation and removal of EEG record areas with individually determined parameters of eye movement artefacts—according to the exceedance of the threshold amplitude;
2. Removal of muscular artefacts—the removal of EEG record areas with a high-amplitude high-frequency component.

To remove eye movement artefacts automatically, an EEG is registered for minimum 10 seconds, during which operator 100 is instructed to blink freely several times. According to this record, an average blinking amplitude is determined in the selected channel, as a rule, in channels Fp1 and/or Fp2, and average blinking time. Based on the calculated amplitude a threshold is set, the exceedance of which is considered a sign of an artefact. To determine artefacts automatically, a 60% threshold is set from the peak value at the test area with artefacts (in assignments Fp1, Fp2); the period of eye movement artefact is time from the blinking peak to the second cross of the signal with the isoline. When the exceedance of the threshold in the threshold amplitude is detected during the procedure of artefact removal, the algorithm checks, how many samples (single imaginary movements) are affected by blinking, and marks the current and, if necessary, the following sample as artefactual (the latter case for the situation, if the artefact occurred at the border of two samples).

To remove muscular artefacts, the system accepts the following parameters: frequency range and threshold amplitude. Fourier transformation is calculated for every EEG channel, and the amplitude values are checked in the selected frequency range. In case the amplitude is exceeded, the sample is marked as artefact and is excluded from the further analysis.

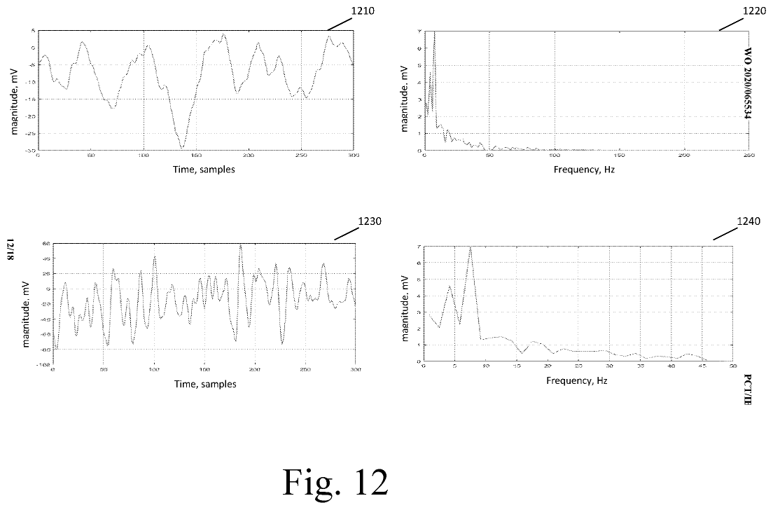

FIG. 12 shows a flowchart of the signal in one of EEG recording channels—channel T5 without muscular artefacts 1210 and frequency distribution 1220, corresponding to this signal. For reference, FIG. 12 also shows a flowchart with muscular artefacts in channel T5 1230, and the result of Fourier transformation for this signal 1240.

In 20-35 Hz interval in the sample with muscular artefacts, the signal amplitude is several times bigger than that in the sample without artefacts. With the amplitude values in the given frequency range, the presence or muscular artefacts in the signal is determined in real time mode.

To increase spatial resolution and to detect informative characteristics of an EEG in the developed EEG signal registration system, several transformations can be implemented, i.e. spatial filters: reduction to common average montage, weighted average referent montage and transformation into current source density. The implementation of several approaches focuses on the possible use of individual system configuration algorithms.

The above-mentioned EEG registration system allows to simultaneously perform registration, synchronization, transformation and processing of EEG signals in time and frequency domains. To decrease response time of the system during data processing in real time, the following approaches are used: applying signal filtration and signal preliminary processing with minor time for parameter calculation; decreasing the input data domain, decreasing the applied number of informative features; EEG is registered from all the channels, and the calculation of features for classification is made for 2 channels, selected in the result of preliminary analysis. To optimize spatial information, of all EEG channels only several channels are selected, which have informative features. In this project, a set of informative channels is used based on preliminary configuration and mapping of recognition accuracy of imaginary movements, which allows decreasing time for calculation of features and total response time of the system.

According to the main trends of advanced developments, a joint accounting of several spaces of features is implemented in time and frequency domains: the area of segments and the envelope length of EEG signal, wavelet decomposition (discrete and continuous) coefficients, cepstral transformation coefficients. The use of these features is focused on the increase of unification and accuracy of EEG patterns of imaginary movements, on saving minor calculation resources and operation time in online mode.

Throughout the project, the possibility of feedback was implemented (information on accuracy/inaccuracy of the generated tested motor command) with time delay of max. 250 ms, which created the possibility to use this EEG registration system for the development of after-care software in real time mode.

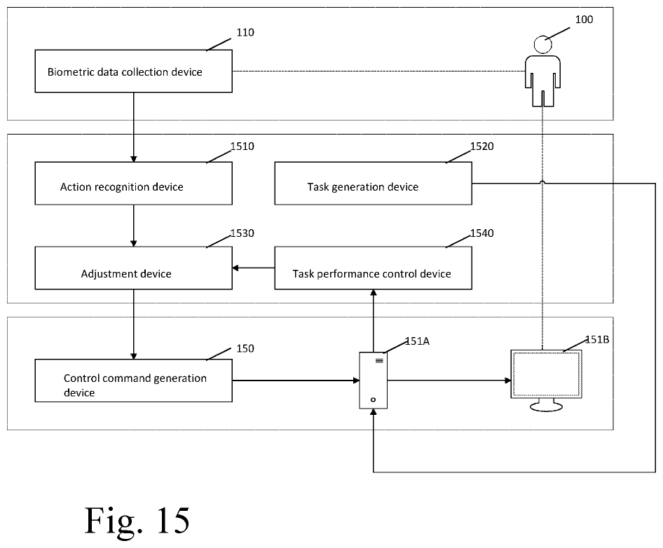

FIG. 15 is an example of the flowchart of operator's after-care system.

A flowchart of operator's after-care system consists of operator 100, bioelectrical data collection means (device) 110, control command generation means (device) 150, calculation center 151A, visualization means (device) 151B, action recognition means (device) 1510, task generation means (device) 1520, adjustment means (device) 1530, task performance control means (device) 1540.

The described system is designed for after-care of people with brain damage or injuries, which result in decreased or disturbed physical activity (for example, people with previous stroke), limb loss (for example, arm loss). Its basic purpose is to stimulate the brain activity or the nervous system activity and flexibility. For this, operator 100 is given tasks, which they must perform, using the system described above in FIG. 1-FIG. 4. At this, the described system adjusts the actions by operator 100, increasing the complexity, thus making increased the activity, i.e. increase stimulation, flexibility and training of the brain and the nervous system.

Action recognition means (device) 1510 is designed to:
Recognize actions performed by operator 100 and calculate data, characterizing the recognizable actions;
Transfer data on actions performed by operator 100 to adjustment means (device) 1530

Action recognition means (device) 1510 is a part of the system, described in FIG. 1, FIG. 2 and contains feature extraction means (device) 120, feature extraction rules base 121, action pattern classification means (device) 130, action pattern classification model 131, action pattern base 132, overtraining means (device) 140.

Task generation means (device) 1520 is designed to:
Generate at least one task, which must be performed by operator 100 by using the described system (including bioelectrical data collection means (device) 110, action recognition means (device) 1510, command generation means (device));
Transfer the generated task to calculation center 151A.

In one of the embodiments, the following act as tasks:
Positioning tasks, in which operator 100 must give commands on moving objects (including virtual objects);
Management tasks, in which operator 100 must give commands on changing the state of an object (for example, activation/deactivation);
Control tasks, in which operator 100 must give commands on maintaining the state according to the configured state or on positioning an object in the given domain.

For example, operator 100 must manage the mouse cursor movement (i.e. give commands on changing the cursor position) on display screen 151B so that the cursor would move on the path, which is pre-configured and marked on display screen 151B.

In another embodiment, operator 100 must paint objects in a configured color, managing the changes (i.e. giving commands on discrete change) of values of color components (for instance, adjusting hues, saturation and lightness), thus operating colors in HSL-color space model).

In another embodiment, operator 100 must hold the cursor on display screen 151B in its original position, while the cursor constantly tries to shift, compensating adjustments by operator 100

So, the main purpose of the generated tasks is to perform interaction of operator 100 and control objects, while feedback is created between operator 100 and control objects so that not only actions performed by operator 100 would affect the state of control object, but changes in the state of control objects would affect operator 100.

In one of the embodiments, the solution of generated tasks is formed and implemented as a gameplay, in the result of which:
Points are given for a successful solution (completeness);
The process of task solving affects the complexity of the current or generated tasks. In one of the embodiments, generation of a new task at least depends on the following:
Which type of activity must be trained;
Which type of tasks is better (faster, easier, with a smaller number of errors) is solved by operator 100 (for example, for regular training of various forms of activity of operator 100).

In one of the embodiments, the following is additionally calculated in generating tasks:
Idealized control commands (examples of actions to train classifiers), which must generate control command generation means (device) 150 for the successful performance of the current task;
System behavior (actions performed by operator 100, action patterns etc.) for the successful performance of the current task.

The above-mentioned calculations can be further used to evaluate the accuracy of the task performed by operator 100.

Adjustment means (device) 1530 is designed to:
Modify the parameters of the identified action patterns (recognized intelligent commands) based on data provided by task performance control means (device) 1540
Transfer the modified action patterns to control command generation means (device) 150.

The main purpose of adjustment means (device) 1530 is to provide feedback between actions performed by operator 100 (by commands given by operator 100) and actions, performed by calculation center 151 A, based on commands, generated by control command generation means (device) 150.

Adjustment means (device) 1530 modifies the parameters of identification action patterns (which affect commands, generated by control command generation means (device) 150) at least for the following:
To decrease the number of errors made by operator 100 to provide regular training process for operator 100;
To help operator 100 to generate commands, i.e. to reduce requirements for action commands (for instance, to increase the range of values, which can accept parameters of action commands), in case if the satisfactory performance of the set task was not reached at some stages by operator 100;
Prevent operator 100 from generating commands, i.e. increase requirement to action commands (for instance, to decrease the range of values, which can accept parameters of action commands), in case if any stages of task solving were easily run by operator 100, which did not have any training effect.

For example, operator 100 is given the task to move the cursor on some curve (for instance, on a vertical straight line in easy mode, and on a quadrifoil in a hard mode), so that the maximal distance between the cursor and the curve would not exceed a certain preconfigured task. If operator 100 manages to keep this critical distance, an adjustment is made (identification pattern parameters are modified) so that the cursor would appear at a preconfigured distance, and it would be easier for operator 100 to solve the task (i.e. if operator 100 is not able to perform this task at the moment, which leads to overfatigue and loss of training effect, the task must be made easier). If operator 100 manages to keep not only the mentioned critical distance, but a smaller distance (i.e. operator 100 solves the current task successfully), an adjustment is made so that the cursor would appear at a critical distance, and it would be harder for operator 100 to solve the task (i.e. operator 100 can easily solve the current task at the moment, which leads to less fatigue than that required for training).

In another embodiment, the adjustment can be implemented as follows:
For strong/successful operator 100 (operator 100, who solves the task easily), if the shift of the cursor from the curve is Dc, an adjustment is performed to make the cursor shift to the opposite site at the value of 3×Dc; thus, the cursor swings along the curve, making operator 100 wish to decrease the shift from the curve Dc;
For weak/unsuccessful operator 100 (operator 100, who hardly solves the task), if the shift of the cursor from the curve is Dc, an adjustment is performed to make the cursor shift to the opposite site at the value of 0.75×Dc, thus, the cursor is pressed to the curve, making it easier for operator 100 to perform the task to wish to decrease the shift from the curve Dc.

Task performance control means (device) 1540 is designed to:
Analyze the performance by operator 100 of the task, generated by task generation means (device) 1520, based on data, acquired from calculation center 151A;
Calculate data, describing the adjustment of actions performed by operator 100, based on the results of the performed analysis;
Transfer the calculated data to adjustment means (device) 1530.

In one of the embodiments, the following acts as performance analysis of the task by operator 100:
The comparison of control command parameters, generated by control command generation means (device) 150, with the parameters of idealized control command, calculated by task generation means (device) 1520 when generating the task;
The comparison of the result of the performance of control command, generated by control command generation means (device) 150, with the expected result, calculated by task generation means (device) 1520 when generating the task.

Calculation center 151A is designed to:
Analyze the task, acquired from task generation means (device) 1520;
Display information on the task for operator 100 with visualization means (device) 151B;
Monitor the performance of the task by operator 100;
Transfer data on the monitored process to task performance control means (device) 1540

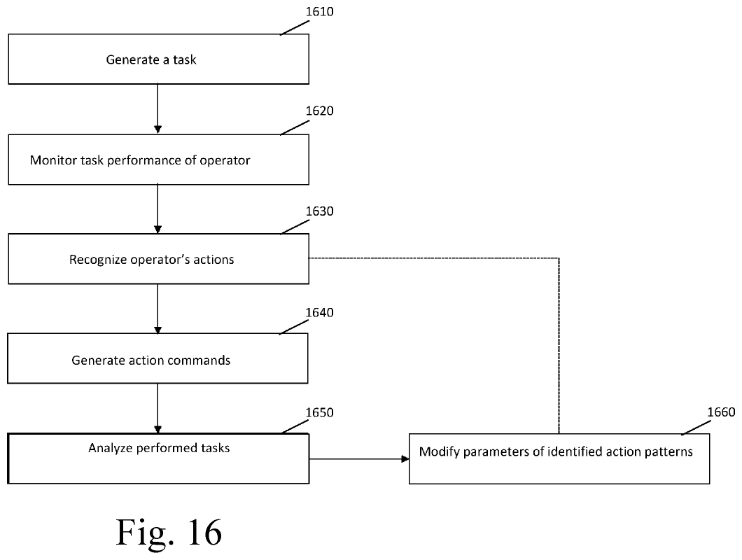

FIG. 16 is an example of the flowchart of operator's after-care method.

A flowchart of operator's after-care method consists of stage 1610, at which a task is generated, stage 1620, at which task performance by operator is monitored, stage 1630, at which actions by operator are recognized, stage 1640 at which action commands are generated, stage 1650, at which task performance is analyzed, stage 1660, at which parameters of identified action patterns are modified.

At stage 1610, task generation means (device) 1520 is used to generate at least one task, which operator 100 must perform using the described system (including bioelectrical data collection means (device) 110, action recognition means (device) 1510, command generation means (device));

At stage 1620, calculation center 151 A is used to:
Display information on the task for operator 100 with visualization means (device) 151B;
Monitor the process of task performance by operator 100.

At stage 1630, action recognition means (device) 1510 is used to recognize the actions performed by 100 and to calculate data, characterizing the recognizable actions.

At stage 1640, control command generation means (device) 150 is used to generate action commands to solve the set task.

At stage 1650, task performance control means (device) 1540 is used to:
Analyze the performance by operator 100 of the task, generated at stage 1610, based on data acquired from calculation center 151A;
Calculate data, describing the adjustment of actions performed by operator 100, based on the results of the performed analysis.

At stage 1660, adjustment means (device) 1530 is used to modify the parameters of identified action patterns based on data calculated at stage 1650.

Stages 1620-1660 can be performed until the following at least occurs:
A generated task is performed;
A number of errors, made by operator 100, reaches the preconfigured value;
The configured time is over.

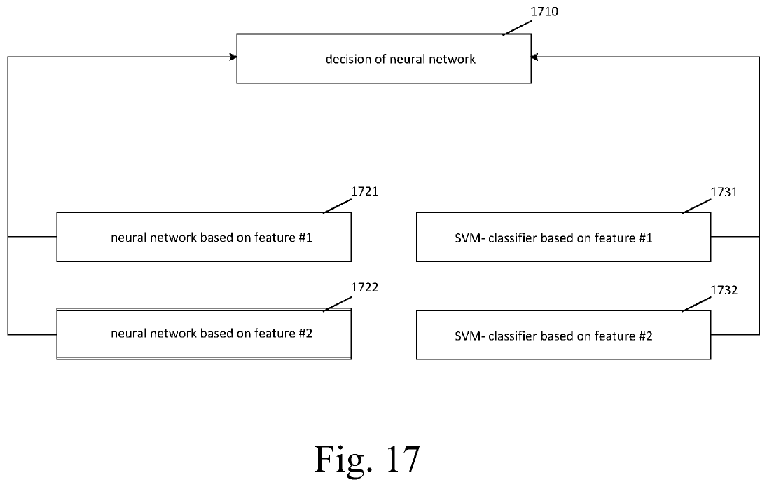

FIG. 17 is an example of the flowchart of classifiers' committee.

A flowchart of classifiers' committee contains decision neural network 1710, neural network based on feature #1 1721, neural network based on feature #2 1722, SVM-classifier based on feature #1 1731, SVM-classifier based on feature #2 1732.

In order to recognize imaginary actions according to EEG, a committee of classifiers is implemented, which is based on support vector machines and artificial neural networks (FIG. 14). These approaches are effective classification methods, particularly for multichannel EEG signals.

The support vector machines method belongs to linear classification methods. The essence of the method is the separation of the sample into classes with optimal separating hyperplane, the equation of which in the general case is as follows: f(x)=(ω,(x))+b,

where ω=Σᵢ₌₁^{N}λᵢyᵢ(xⱼ),
coefficients λᵢ depend on yᵢ (vectors of class marks) and on the value of scalar products ((xᵢ), (xⱼ)). Thus, to find a decision function, it is necessary to know the values of scalar products. Data transformations are determined by the kernel function: K(x,y)=((x), (y)).

Base on the study results on the selection of a preferable SVM type to classify EEG-signals, Gaussian radial basic function SVM-RBF SVM is used as a kernel function:

K(xᵢ,xⱼ)=e^{-γx}^{ᵢ}^{-x}^{ⱼ}².

The above mentioned artificial neural networks (ANNs) are based on the principles of distributional, non-linear and parallel data processing with learning. In the given paper, ANNs are implemented in the form of a multi-layer perceptron, consisting of three layers: two discrete layers and one output layer. A sigmoid function is used as a function to activate in discrete layers

$$out = \frac{1}{1 + {e}^{- α Y}} ,$$

where α—a slope parameter of the sigmoid function, and a linear function in the output layer.

For the purpose of simultaneous account of several types of features, a 2-level committee of local classifiers is used, the lower level of which consists of 2 ANNs and 2 support vector machines. The upper level consists of an ANN, which unites the classification results of the lower level (FIG. 17).

The following parameters are used as features: area under the curve, the curve complexity, discrete and continuous wavelet decomposition coefficients. Lower-level classifiers input the features of various types and decide on classification of the given EEG signal. These decisions are summarized into a vector and are input to the ANN of the upper level, which performs the final classification, i.e. relates the analyzed EEG signal to one of the classes (FIG. 14). Thus, there is a possibility to select the best features for classification.

The upper-level ANN is trained on a dataset, including the solutions from the lower-level local classifiers. The trained upper-level ANN defines the importance of the solutions of every lower-level classifier and performs the selection of the best solution.

Due to the implemented structure, the software of the BCI platform can be individually configured for the user, allowing the selection of the most relevant features, whereas the committee of classifiers is easily scaled, including new lower-level classifiers.

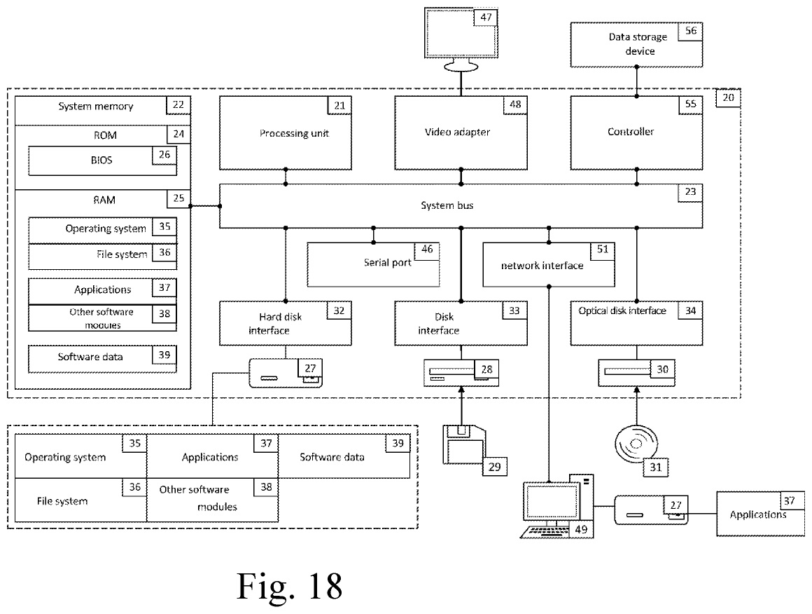

FIG. 18 is an example of general-purpose computing system: personal computer or server 20 with central processing unit 21, system memory 22 and system bus 23, which contains various system components, including memory connected with central processing unit 21. System bus 23 is implemented as any bus structure known in the prior art, which in its turn contains bus memory or a bus memory controller, a peripheral bus and a local bus, which can interact with any other bus architecture. System memory contains read-only memory (ROM) 24, random access memory (RAM) 25. Basic input/output system (BIOS) 26, contains the main procedures, which provide information transfer between the elements of personal computer 20, for example, at the moment of operating system loading using ROM 24.

In its turn, personal computer 20 contains hard disk drive 27 to read and write data, disk drive 28 to read and write data to/from removable disks 29 and optical drive 30 to read and write data to/from optical disks 31, such as CD-ROM, DVD-ROM and other optical data storage means (device)s. Hard disk drive 27, disk drive 28, optical drive 30 are connected with system bus 23 though hard disk interface 32, disk interface 33 and optical drive interface 34, correspondingly. The drives and corresponding computer data storage means (device)s are nonvolatile storage means (device)s for computer instructions, data structures, software modules and other data from personal computer 20.

The given description discloses the implementation of the system, which uses hard disk drive 27, removable disk 29 and removable optical disk 31, but it will be appreciated that other types of computer data storages 56 can be used, which can store data in the form, accessible for reading by computer (solid-state drives, flash memory cards, digital disks, random access memory (RAM) etc.), which are connected to system bus 23 with controller 55.

Computer 20 has file system 36, where written operating system 35 is stored, as well as additional software applications 37, other software modules 38 and software data 39. A user can input commands and information into personal computer 20 with input means (device) (keyboard 40, mouse pointing means (device) 42). Other input means (device) can also be used (not shown): a microphone, a joystick, a game console, a scanner etc. Such input means (device)s are usually connected to the system of computer 20 with serial port 46, which in its turn is connected to system bus, but they can also be connected in a different way, for example, with parallel port, game port or universal serial bus (USB). Monitor display 47 or any other type of display means (device) is connected to system bus 23 though an interface, such as video display adapter 48. In addition to monitor display 47, a personal computer can be equipped with other peripheral output means (device)s (not shown), for example, speakers, a printer etc.

Personal computer 20 can operate in a networked environment; at that, network connection with one or several remote computers 49 is used. A remote computer (computers) 49 are the same personal computers or servers, which can have all or most of the components, described earlier for the concept of personal computer 20, shown in FIG. 18. A computer network can also have other means (device)s, for example, routers, network stations, peering means (device)s and other net points.

Network connections can form local area network (LAN) 50 and a wide-area network (WAN). Such networks are used in corporate computer networks, internal corporate networks and as a rule they have Internet access. In LAN- or WAN-networks, personal computer 20 is connected to local area network 50 through network adapter or network interface 51. When using networks, personal computer 20 can use modem 54 or other connection assistance means (device)s for global computing network, such as Internet. Modem 54, which is an internal or external means (device), is connected to system bus 23 with serial port 46. It must be mentioned, that network connections are only exemplary and do not have to show the exact network configuration, i.e. in reality there are other ways to make connections of one computer with another using technical means.

In the end, it must be mentioned that the data given in the present description, are examples, which do not confine the scope of the invention under the patent claim.

1.-29. (canceled)

30. A method of real time rehabilitation and training comprising steps of:
a. forming a virtual domain further comprising an operator's character;
b. forming a task to be performed by an operator;
c. collecting operator's bioelectrical data;
d. detecting characteristic features of said collected bioelectrical data by means of artificial intelligence;
e. defining an action pattern according to said detected characteristic features;
f. generating a control command for said virtual domain based on said defined action pattern which is displayed to said operator;
g. Evaluating execution performance of said operator's action;
h. Evaluating operator's task execution performance;
i. Providing a feedback to the operator's executed task in real time;
j. Performing a calibration for the operator.

31. The method of claim 30, wherein said step of collecting said operator's bioelectrical data further comprise collecting at least one of the following:
a. an operator's electroencephalogram being a set of electroencephalographic signals of an operator's nervous system; said set is characterized by a signal registration time of said electroencephalographic signals and a signal amplitude of said electroencephalographic signals; and
b. an operator's electromyogram being a set of electromyographic signals of an operator's muscular system; said set is characterized with a signal registration time of said electromyographic signals and a signal amplitude of said electromyographic signals.

32. The method of claim 30, wherein said step of extracting at least one characteristic features collected from bioelectrical data is performed by means of at least one of the following:
i. a trained model for feature extraction,
ii. a set of feature extraction rules.

33. The method of claim 32, wherein said step of said at least one characteristic feature selected from the group consisting of: a spectral characteristic, a time characteristic, a wavelet decomposition characteristic, a spatiotemporal characteristic and any combination thereof.

34. The method of claim 30, wherein said step of evaluating execution performance comprises evaluating conformity of said state of said virtual object after performing said operation by said operator at said virtual object; said conformity is evaluated in comparison with a predesigned resultant state of said virtual object after performing said operator's action.

35. The method of claim 30, wherein said step of evaluating operator's task execution performance further comprises evaluating a number of errors of performing said action by said operator at the virtual object; said errors are indicated when said action is performed by said operator at the virtual object with an execution performance lower than a preconfigured value.

36. A computer-implemented system for generating control commands based on the operators' bioelectrical data; said system comprising:
a. a processor;
b. a memory storing instructions which, when executed by said processor, direct said processor to:
i. collecting operator's bioelectrical data and transferring said collected data;
ii. extracting at least one characteristic feature from collected bioelectrical data by means of at least one of the following:
1. a trained model for feature extraction based on machine learning, and
2. a set of feature extraction rules;
said at least one characteristic feature is selected from the group consisting of: a spectral characteristic, a time characteristic, a wavelet decomposition characteristic, a spatiotemporal characteristic and any combination thereof;
c. defining an action pattern according to said extracted characteristic features by means of artificial intelligence; said action pattern being a numerical value, which characterizes the possibility of belonging said operator's collected bioelectrical data to said action;
d. generating a control command based on an action pattern.

37. The system of claim 36, wherein said operator's bioelectrical data further comprise at least one of the following:
a. an operator's electroencephalogram being a set of activity signals of an operator's nervous system; said set is characterized by a signal registration time and a signal amplitude thereof; and
b. an operator's electromyogram being a set of activity signals of an operator's muscular system; said set is characterized by a signal registration time and a signal amplitude thereof.

38. The system of claim 36, wherein said instructions comprise extracting at least two samples from said collected bioelectrical data; each said sample is a set of data describing a single image of an operator's move.

39. The system of claim 36, wherein said action pattern is defined by a two-level committee of local classifiers comprising a lower level and an upper level; said lower level further comprises a combination of at least one classifier based on a support vector machine and at least one artificial neural network; said upper level further comprises at least one artificial neural network.

40. The system of claim 39, wherein said artificial neural network of said upper level of committee of local classifiers is trained on a dataset comprising solutions for each of said local classifier of said lower level.

41. The system of claim 36, wherein said memory comprises an instruction of analyzing and transforming said collected data; said instruction of analyzing and transforming said collected data further comprises:
a. applying high and low frequency filters;
b. removing a network noise by applying at least one of band elimination and band-pass filters,
c. filtering filtered EEG signals;
d. transforming said EEG signal into mean, weighed mean composition, current source density, topographies of independent components.

42. The system of claim 36, wherein said instructions comprise an instruction of forming of an image of said action and displaying thereof to said operator.

43. The system of claim 36, wherein said instructions comprise simultaneously accounting for the properties of a two-level committee of local classifiers; said two-level committee comprises a lower level further comprising at least two artificial neural networks and at least of two support vector machines, and an upper level comprising an artificial neural network combining classification results of said lower level.

44. A computer-implemented method of generating control commands based on operator's bioelectrical data; said method comprising steps of:
a. providing a computer-implemented system for generating control commands; said system comprising a processor and a memory for storing instructions for implementing said method;
b. collecting operator's bioelectrical data;
c. extracting at least one characteristic features from collected bioelectrical data by means of at least one of the following:
i. a trained model for feature extraction,
ii. a set of feature extraction rules;
said at least one characteristic feature selected from the group consisting of:
a spectral characteristic, a time characteristic, a wavelet decomposition characteristic, a spatiotemporal characteristic and any combination thereof;
d. defining an action pattern according to said extracted features by means of artificial intelligence; said action pattern being a numerical value, which characterizes the possibility of belonging said collected bioelectrical data to said configured imagine action of the operator;
e. generating a control command based on an action pattern.

45. The method of claim 44, wherein said operator's bioelectrical data further comprise at least one of the following:
a. an operator's electroencephalogram being a set of electroencephalographic signals of an operator's nervous system; said set is characterized by a signal registration time of said electroencephalographic signals and a signal amplitude of said electroencephalographic signals; and
b. an operator's electromyogram being a set of electromyographic signals of an operator's muscular system; said set is characterized with a signal registration time of said electromyographic signals and a signal amplitude of said electromyographic signals.

46. The method of claim 44 comprising extracting at least two samples from said collected bioelectrical data; each said sample is a set of data corresponding to a single image of an operator's move.

47. The method of claim 44, wherein said action pattern is defined by a two-level committee of local classifiers, in which the lower level comprises a combination of at least one classifier based on a support vector machine and at least one artificial neural network, and the upper level comprises at least one artificial neural network.

48. The method of claim 47, wherein said artificial neural network of the upper level of committee of local classifiers is trained on a dataset comprising the solutions for each said local classifiers of said lower level.

49. The method of claim 44 comprising steps of analyzing and transforming said collected data; said steps of analyzing and transforming said collected data comprises at least one of the following:
a. applying high and low frequency filters;
b. removing a network noise is removed, using at least band elimination and band-pass filters,
c. filtering EEG signals;
d. transforming EEG signals into mean, weighed mean composition, current source density, topographies of independent components.

50. The method of claim 44 comprising an instruction of forming an image of said action and displaying thereof to said operator.

51. The method of claim 44, wherein a two-level committee of local classifiers is used to simultaneously account for the features; the lower level contains at least two artificial neural networks and at least of two support vector machines, and the upper level contains an artificial neural network, which joint the classification results of the lower level.

52. A computer-implemented system for evaluating execution performance of an operator based on the operator's bioelectrical data; said system comprising:
a. a processor;
b. a memory storing instructions which, when executed by said processor, direct said processor to:
i. generating a virtual domain comprising at least one virtual object characterized by a feature selected from the group consisting of: a position in the virtual domain, a dimension, a color, an interaction rule for said virtual domain, a rule of changing a state of said virtual object depending on an operator's action in said virtual domain;
ii. at least one action to be performed by said operator and related to at least one virtual object;
c. an actuator configured for performing said at least one operator's action under said generated control command in said virtual domain;
said memory further comprises instructions to:
1. Evaluating conformity of said state of said virtual object after performing an action at said virtual object by said operator to a predesigned resultant state of said virtual object after performing said operator's action;
2. Evaluating a number of errors of performing said action by said operator at said virtual object.

53. The system of claim 52, wherein said errors are indicated when said action is performed by said operator at the virtual object with an execution performance lower than a preconfigured value.

54. The system of claim 52, wherein said virtual domain, said virtual objects in the virtual domain and said actions performed by the operator in the virtual domain are visualized.

55. The system of claim 52, wherein said operator's operation comprises a change of said state of said at least one virtual object with said at least one operator's action.

56. The system of claim 55, wherein said change of said state of said at least one virtual object is performed by the operator at least one of the following conditions:
a. within a preconfigured time period,
b. With the preconfigured number of tries.

57. A computer-implemented method of evaluating execution performance of an operator based on the operator's bioelectrical data; said method comprising steps of:
a. providing a computer-implemented system for evaluating execution performance of an operator based on the operator's bioelectrical data; said system comprising a processor and a memory for storing instructions for implementing said method;
b. generating a virtual domain further comprising at least one virtual object; a state of said virtual object having a characteristic selected from the group consisting of: a position in said virtual domain, a dimension, a color, an interaction rule for said virtual domain, a rule of changing said object depending on said operator's action in said virtual domain and any combination thereof; at least one action related to at least one virtual object to be performed by said operator;
c. collecting operator's bioelectrical data;
d. generating at least one control command based on the collected the operator's bioelectrical data;
e. performing at least one operator's action under a generated control command in said virtual domain;
f. evaluating conformity of said state of said virtual object after performing said operation by said operator at said virtual object to a predesigned state of said virtual object after performing said operator's action.
g. evaluating a number of errors of performing said action by said operator at the virtual object.

58. The system of claim 57, wherein said errors are indicated when said action is performed by said operator at the virtual object with an execution performance lower than a preconfigured value.

59. The system of claim 57, wherein said virtual domain, said virtual objects in said virtual domain and said actions performed by said operator at the virtual domain are visualized.

60. The method of claim 57, wherein said operator's operation comprises a change of said state of at least one of said virtual object with at least one of said operator's action.

61. The method of claim 60, wherein said change of said state of said virtual object is performed by said operator at least one of the following conditions:
a. within a preconfigured time period,
b. With a preconfigured number of tries.
